(19) United States
(12) Patent Application Publication
(10) Pub. No.: US 2014/0088632 A1
(43) Pub. Date: Mar. 27, 2014

(54) LANCET DEVICE UTILIZING A MAGNET AND METHOD OF MAKING AND USING THE SAME

(71) Applicant: STAT MEDICAL DEVICES, INC., N. Miami Beach, FL (US)

(72) Inventors: James BOOKER, Leominster, MA (US); Paul FULLER, Danvers, MA (US); David CARHART, Cary, IL (US); Frank PISTORIO, Itasca, IL (US); Oleksiy DIDKOVSKYY, Lviv (UA); Nazar PELENJO, Lviv (UA); Steven SCHRAGA, Surfside, FL (US)

(73) Assignee: STAT MEDICAL DEVICES, INC., N. Miami Beach, FL (US)

(21) Appl. No.: 13/784,247

(22) Filed: Mar. 4, 2013

Related U.S. Application Data
(60) Provisional application No. 61/704,163, filed on Sep. 21, 2012.

(51) Int. Cl. A61B 5/151

(52) U.S. Cl. CPC A61B 5/15117
USPC 606/182

(57) ABSTRACT

Lancet device includes a magnet member, a sleeve member capable of being affected by magnetic forces emanating from the magnet member, and a movable lancet holder. At least one of the following is also utilized; a lancet releaser movable at least between an initial position and a position that allows a lancet to be removed from the lancet holder and a lancet holder lock movable at least between a lock position that prevents axial movement of the lancet holder along an axial direction and an unlock position that allows the lancet holder to move along the axial direction.

### CROSS-REFERENCE TO RELATED APPLICATIONS

The instant application is a US non-provisional application based on U.S. provisional application No. 61/704,163 filed Sep. 21, 2012, the disclosure of which is hereby expressly incorporated by reference hereto in its entirety.

### BACKGROUND OF THE INVENTION

1. Field of the Invention

The present invention generally relates to a lancet device, and more particularly, to a lancet device utilizing a magnetic drive/retraction system.

2. Discussion of Background Information

Lancet devices are commonly used to prick the skin of the user so that one or more drops of blood may be extracted for testing. Some users, such as diabetics, for example, may have to test their blood sugar levels several times a day. This may be accomplished by the user using a simple needle. However, this procedure is often problematic for the user since the needle may be difficult to handle. Moreover, controlling the depth of penetration cannot be reliably accomplished without the use of a mechanical device. Additionally, many users simply cannot perform the procedure owing to either a fear of needles or because they lack a steady hand. As a result, lancet devices have been developed which allow the user to more easily and reliably perform this procedure.

Many lancet devices lack convenient and flexible adjustability. Some devices are typically made adjustable by switching their tips. U.S. Pat. No. Re. 32,922 to LEVIN et al. is one such device. That is, the user must remove one tip having a set depth and replace it with another having a different set depth. This, of course, creates the problem of storing the replaceable tips, which if not properly done, may result in their misplacement, damage, contamination, or the like.

An improved device would allow the user to more easily adjust the depth of penetration and would overcome some of the disadvantages described above. Moreover, since the skin thickness can vary slightly from user to user and finger to finger, a need exists for efficiently adapting the depth of penetration. For example, an index finger may be more calloused than a middle finger, and the more calloused finger will typically have thicker skin. By adjusting the depth of puncture so that the depth is no greater than necessary for extracting a required amount of blood, any pain experienced by the user may be minimized.

Lancets having an adjustable tip are known per se. For example, U.S. Pat. No. 4,469,110 to SLAMA discloses a mechanism which adjusts the penetration depth by rotating a threaded sleeve relative to a body. The SLAMA device is characterized as a “single bottom” device which employs a threaded design which can be expensive to manufacture. Moreover, such a device may require the user to rotate the threaded sleeve up to 360 degrees and more in order to attain the proper depth setting. Further, such a threaded resign is prone to inadvertent setting changes since there is nothing but frictional engagement between the mating threads to maintain the adjustment setting.

U.S. Pat. No. 4,895,147 to BODICKY et al. functions in a similar manner to the device in SLAMA and therefore suffers from similar disadvantages.

U.S. Pat. Nos. 5,464,418, 5,797,942, 5,908,434, 6,156,051 and 6,530,937 to SCHRAGA also disclose similar lancet devices and are hereby incorporated herein by reference as though set forth in full herein.

As disclosed in U.S. Pat. No. 5,908,434, the lancet device has a body portion which encloses a lancet and a lancet firing mechanism. The lancet typically has a needle extending therefrom and is caused to move towards the tip of the device by a trigger or firing mechanism. The lancet device forces the needle, by virtue of the needle being fixed thereto, out of the device by some distance or depth so that the needle can penetrate the skin of the user. The function of this firing mechanism and the lancet body design is disclosed in each of U.S. Pat. Nos. 5,797,942 and 5,908,434. These patents are incorporated by reference herein in their entirety and are therefore only briefly discussed herein. Similarly, U.S. Pat. No. 6,156,051 discloses a lancet device which utilizes a lancet firing mechanism, a depth adjustment mechanism, and a trigger setting mechanism. This patent is incorporated by reference herein in its entirety.

What is needed is a lancet device which can accurately and precisely control the depth of penetration of the needle relative to the surface of the user's skin while also being easy to use. It is also desirable for the user to be able to more easily and safely install/remove a lancet from the device. Additionally, it would be advantageous to include additional safety features such as locking or prevent axial movement of the lancet device holder. A magnetic lancet device could offer at least some of these features and have additional advantages.

U.S. 2005/0125019 and U.S. 2005/0125017, both to KUDRNA et al., each disclose a magnetic lancet device which offers some advantages. Although the device disclosed therein offers advantages over prior lancet devices, this device can be significantly improved. These US patent application publications to KUDRNA et al. are incorporated by reference herein in their entireties and are therefore only briefly discussed herein.

While advances have been made in improving lancet devices, there is a continuing need for a lancet device which provides for additional advantages relating to ease of use, safety, reliability and repeatability.

### SUMMARY OF THE INVENTION

In accordance with one non-limiting embodiment of the invention there is provided a lancet device comprising a magnet member, a sleeve member capable of being affected by magnetic forces emanating from the magnet member and a movable lancet holder. At least one of the following is also utilized; a lancet releaser movable at least between an initial position and a position that allows a lancet to be removed from the lancet holder and a lancet holder lock movable at least between a lock position that prevents axial movement of the lancet holder and an unlock position that allows the lancet holder to move axially.

In embodiments, the lancet holder is movable to a retracted position or cocked position and an extended position or a piercing position.

In embodiments, the sleeve member is axially movable at least between an initial position and a retracted position and an extended position and the initial position.

In embodiments, the sleeve member is a generally cylindrical metal member.

In embodiments, the magnet member is a generally cylindrical permanent solid magnet member.

In embodiments, the lancet device further includes a slide button movable at least between at least one of: an initial position to a cocking or arming position; a first position defined by a front portion of the slide button being generally adjacent a proximal end of the lancet device and a second position defined by a rear portion of the slide button being generally adjacent a distal end of the lancet device; an initial position to a triggering position; and an initial position to an arming and triggering position.

In embodiments, the lancet device further comprises an axially movable arming/triggering device that is movable at least between a first position and a second position.

In embodiments, in the first position, the arming/triggering device engages with a surface movable with the lancet holder, and, in the second position, disengages with the surface.

In embodiments, the arming/triggering device comprises a first member that is movable via a slide button and a second member with the first member and movable relative to the first member\.

In embodiments, the lancet device further comprises an axially movable sleeve assembly that is movable at least between a first position and a second position.

In embodiments, in the first position, at least one projection of the sleeve assembly engages with a surface movable with the lancet holder, and, in the second position, disengages with the surface.

In embodiments, the sleeve assembly comprises a first member that is movable via a slide button and a second member with the first member and movable relative to the first member.

In embodiments, the magnet member is at least one of: axially retained, fixed in place within the body, has an axial length less than the axial length of the sleeve member; and is coupled to the body via a member.

In embodiments, the lancet device comprises each of: the lancet releaser movable at least between an initial position and a position that allows the lancet to be removed from the lancet holder; and the lancet holder lock movable at least between a lock position that prevents axial movement of the lancet holder along an axial position an unlock position that allows the lancet holder to move along the axial position.

In embodiments, the lancet releaser comprises at least one of: a slide member that can move at least partially circumferentially; a projection that engages with a projection arranged on the lancet holder; and a torsion spring.

In embodiments, the lancet holder lock comprises at least one of: an axially movable member; a pivotally mounted projection that engages with a recess or slot arranged on the lancet holder; at least one projection adapted to moved when a front cap of the lancet device is installed; and a compression spring.

In embodiments, the lancet device further comprises a spring at least partially surrounding a distal portion of the lancet holder.

In embodiments, the lancet device further comprises a compression spring arranged within a space disposed within the lancet holder.

In embodiments, the lancet device further comprises a compression spring that expands at least one of: axially when a front cap of the lancet device is removed and compresses axially when a front cap of the lancet device is installed.

In embodiments, the lancet device further comprises a torsion spring that biases the lancet releaser towards the initial position.

In embodiments, the lancet device further comprises a front cap installable on a proximal end of the body.

In embodiments, the lancet device further comprises a front cap assembly installable on a proximal end of the body.

In embodiments, the front cap assembly comprises plural components.

In embodiments, the front cap assembly comprises depth adjustment.

In embodiments, the front cap assembly comprises a depth adjustment member.

In embodiments, the body comprises plural components.

In embodiments, the lancet holder comprises plural components.

In embodiments, the lancet releaser comprises plural components.

In embodiments, the lancet holder lock comprises plural components.

In embodiments, the sleeve member is fixed to a portion of the lancet holder.

In embodiments, the lancet device further comprises a lancet adapted to be installed on a front portion of the lancet holder.

In embodiments, the lancet device further comprises an arming/triggering system that includes: a slide button mounted to the body; an arming device releasably engaging with a surface that moves with the lancet holder; and a spring biasing at least one of the slide button and the arming device towards an initial position.

In embodiments, the lancet device further comprises an arming/triggering system that includes: a slide button mounted to the body and having a first portion disposed inside the body and a second portion arranged outside the body; an arming sleeve assembly comprising at least one projection releasably engaging with an opening that moves with the lancet holder; and a spring biasing at least one of the slide button and the arming sleeve assembly towards an initial position.

In embodiments, the lancet device further comprises an arming/triggering system that includes: a slide button mounted to the body and having a projection disposed inside the body and a finger engaging portion arranged outside the body; an arming sleeve comprising a projection engageable by the projection of the slide button and at least one projection releasably engaging with an opening that moves with the lancet holder; and a compression spring biasing the arming sleeve towards an initial position.

In accordance with one non-limiting embodiment of the invention there is provided a method of using the lancet device of any of the types described above, wherein the method comprises moving the sleeve member from a first position surrounding the magnet member to a second more distal position and after the lancet holder moves to toward an extended position, moving the sleeve member back to the first position surrounding the magnet member.

In accordance with one non-limiting embodiment of the invention there is provided a method of using the lancet device of any of the types described above, wherein the method comprises manually moving the sleeve member from a first position surrounding the magnet member to a second more distal position and after the lancet holder moves to toward an extended position, automatically moving the sleeve member back to the first position surrounding the magnet member.

In accordance with one non-limiting embodiment of the invention there is provided a method of using the lancet device of any of the types described above, wherein the method comprises moving the sleeve member from a first position surrounding the magnet member to a second more distal position via a slide button and after the lancet holder moves to toward an extended position, allowing the magnet member to move the sleeve member back to the first position surrounding the magnet member.

In accordance with one non-limiting embodiment of the invention there is provided a method of using the lancet device of any of the types described above, wherein the method comprises moving the lancet holder with the sleeve member connected thereto from a first position wherein the sleeve member surrounds the magnet member to a second more distal position, releasing the lancet holder so that it moves to toward an extended position, and allowing the sleeve member to move back to the first position surrounding the magnet member, wherein the extended position is determined by magnetic forces produced by the magnetic member acting on the sleeve member.

In accordance with one non-limiting embodiment of the invention there is provided a method of using the lancet device of any of the types described above, wherein the method comprises removing a front cap from the lancet device and moving a lancet release button to cause a lancet to release from the lancet holder.

In embodiments, the method includes after the removing, automatically preventing axial movement of the lancet holder.

In accordance with one non-limiting embodiment of the invention there is provided a method of using the lancet device of any of the types described above, wherein the method comprises removing a front cap from the lancet device and after the removing, moving a locking member into engagement with the lancet holder.

In accordance with one non-limiting embodiment of the invention there is provided a method of using the lancet device of any of the types described above, wherein the method comprises removing a front cap from the lancet device and after the removing, automatically moving a locking member into engagement with the lancet holder.

In accordance with one non-limiting embodiment of the invention there is provided a method of using the lancet device of any of the types described above, wherein the method comprises installing a front cap on the lancet device, wherein during the installing, a locking member is moved out of engagement with the lancet holder.

In accordance with one non-limiting embodiment of the invention there is provided a method of using the lancet device of any of the types described above, wherein the method comprises installing a front cap on the lancet device, wherein during the installing, a locking member is automatically moved out of engagement with the lancet holder.

In accordance with one non-limiting embodiment of the invention there is provided a method of making the lancet device of any of the types described above, wherein the method comprises axially fixing the magnetic member within the body and mounting the sleeve member to a portion of the lancet holder, wherein the lancet device comprises depth of penetration adjustment, manual or user activated lancet release, and automatic lancet holder locking.

In accordance with one non-limiting embodiment of the invention there is provided a method of making the lancet device of any of the types described above, wherein the method comprises axially fixing the magnetic member within the body, mounting the sleeve member to a portion of the lancet holder, and mounting a slide button to the body, wherein the lancet device is automatically triggered upon movement of the slide button to a predetermined location, and wherein the lancet device comprises depth of penetration adjustment, manual or user activated lancet release, and automatic lancet holder locking.

In accordance with one non-limiting embodiment of the invention there is provided a lancet device utilizing a more quiet movement of the lancet holding member including movement toward the extended position and movement to a retracted position. The quietness of the movement can be characterized as whisper quiet. The extended position can be said to be said to be a limit of magnetic travel. Moreover, by utilizing magnetic forces to regulate the maximum extended position as well as a retracted position, no hard stops or mechanical contact or impact forces or noises need be utilized. Elimination of such hard stops reduces the noise generated by triggering the lancet device. Moreover, no springs need to be utilized to cause the movement of the holding member to the extended position and/or the retracted position. Finally, it is believed that because essentially no (or little) sound occurs upon triggering or activation, less pain is felt (a psychological effect) when skin puncturing occurs.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIGS. 1-6 show various view of a lancet device in accordance with the invention;

FIG. 7 shows a side cross-section view of the lancet device of FIG. 1 in an initial or ready-to-use position;

FIG. 8 shows a side cross-section view of the lancet device of FIG. 1 in cocked and about to trigger position;

FIG. 9 shows a side cross-section view of the lancet device of FIG. 1 in a triggered position with the lancet in a fully extended or puncturing position;

FIGS. 10 and 11 show various side cross-section views of the lancet device of FIG. 1 in a triggered position with the lancet in a fully extended or puncturing position and with the cap located in the lowest or shallowest puncturing depth position;

FIGS. 12-19 show various line drawing views of the left-side housing or body portion of the lancet device of FIG. 1;

FIGS. 20-27 show various line drawing views of the right-side housing or body portion of the lancet device of FIG. 1;

FIGS. 28-66 show various views of the cap or front cover assembly portion of the lancet device of FIG. 1 and the components used therein or thereon;

FIGS. 67-72 show various views of the slide or firing button portion of the lancet device of FIG. 1;

FIGS. 73-103 show various views of the lancet holder assembly/system portion of the lancet device of FIG. 1 which includes, among other things, the lancet holder, the magnet support member, the metal sleeve, the magnet, and the lancet;

FIG. 104 shows side views of a housing half of the lancet device of FIG. 1 with the magnet support member installed thereon;

FIGS. 105-110 show various views of the lancet lock portion of the lancet device of FIG. 1;

FIGS. 111-117 show various views of the lancet plunger portion of the lancet device of FIG. 1;

FIGS. 118-122 show various views of the lancet of the lancet device of FIG. 1;

FIGS. 123-125 show various views of the spacer member portion of the lancet device of FIG. 1;

FIGS. 126-151 show various views of the movable sleeve assembly portion of the lancet device of FIG. 1;

FIGS. 152-161 show various views of the movable sleeve assembly installed on a portion of the assembly shown in FIGS. 73-103;

FIGS. 162-165 show various views of the lancet loading and release system used on the lancet device of FIG. 1;

FIGS. 166-172 show various views of the lancet release button used on the lancet device of FIG. 1;

FIGS. 173-175 show various views of the lancet release spring used on the lancet device of FIG. 1;

FIGS. 176 and 177 show various views of the magnet member used on the lancet device of FIG. 1;

FIGS. 178 and 179 show various views of the metal collar or sleeve member used on the lancet device of FIG. 1;

FIGS. 180 and 181 show various views of the lancet holder lock spring used on the lancet device of FIG. 1;

FIGS. 182 and 183 show various views of the lancet spring used on the lancet device of FIG. 1;

FIGS. 184 and 185 show various views of the lancet device rearming spring used on the lancet device of FIG. 1;

FIGS. 186-189 show various views of the lancet holder lock member used on the lancet device of FIG. 1;

FIGS. 190-196 show various views of the lancet holder lock ring used on the lancet device of FIG. 1;

FIGS. 197-201 show various views of the lancet holder lock system in unlocked and in the activated or locked position used on the lancet device of FIG. 1;

FIG. 202 shows a side cross-section view of the assembled inner workings of the lancet device of FIG. 1; and

FIG. 203 shows various views describing the operation of the lancet holder lock system used on the lancet device of FIG. 1.

### DETAILED DESCRIPTION OF THE INVENTION

The lancet device LD includes a number of components which will be described in detailed below with reference to the drawings. The main components of the lancet device LD include a lancet L, a body 1 formed of body sections 2 and 3, a front cover or cap assembly 10, a slide button 20, a lancet holder assembly 30 and 40, a movable sleeve assembly 50, a lancet release system 60, a holder lock system 70, a magnet 90, a metal sleeve 95, a spacer member SM, a first spring S1, a second spring S2, a third spring S3 and fourth spring S4.

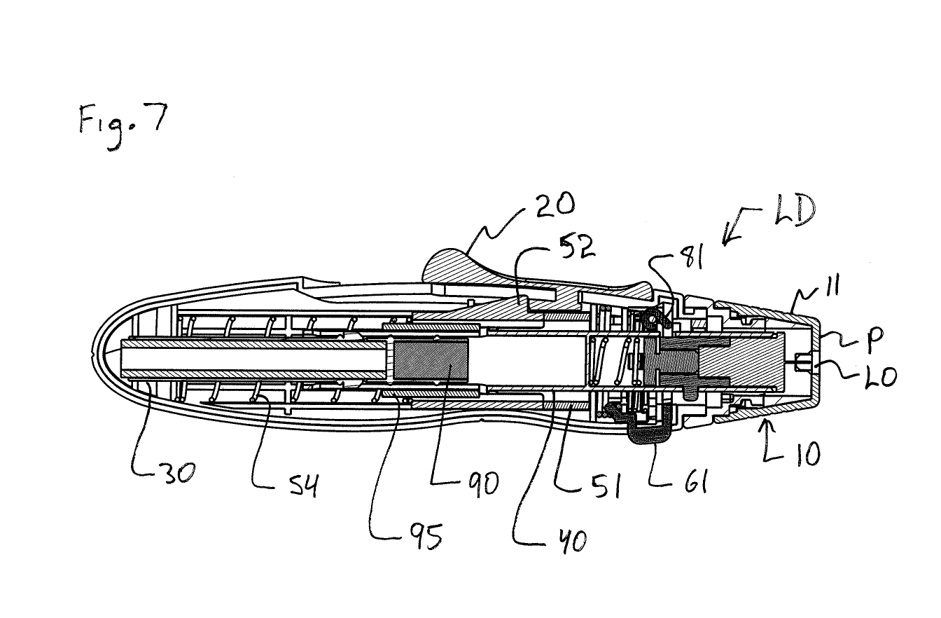

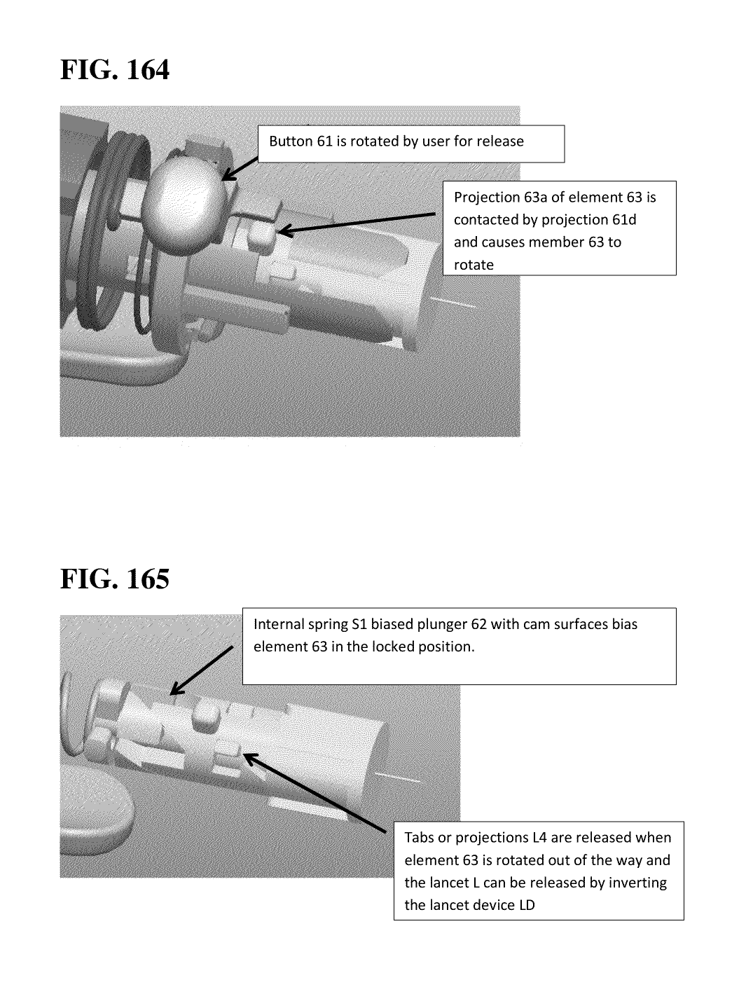

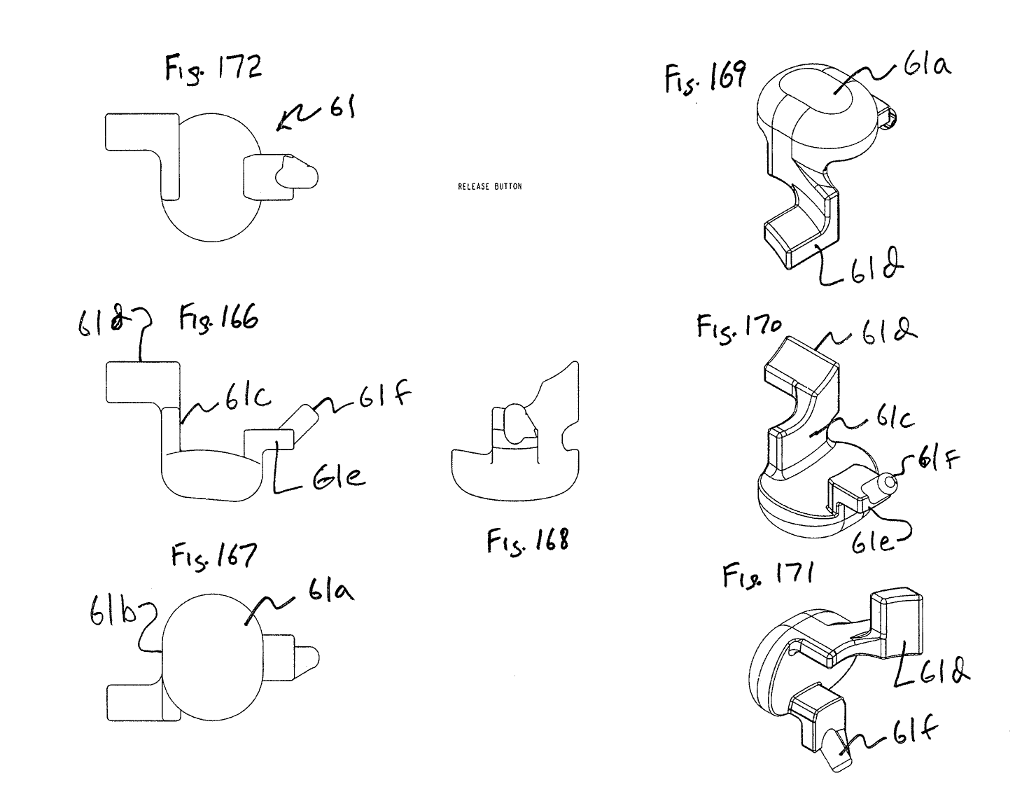

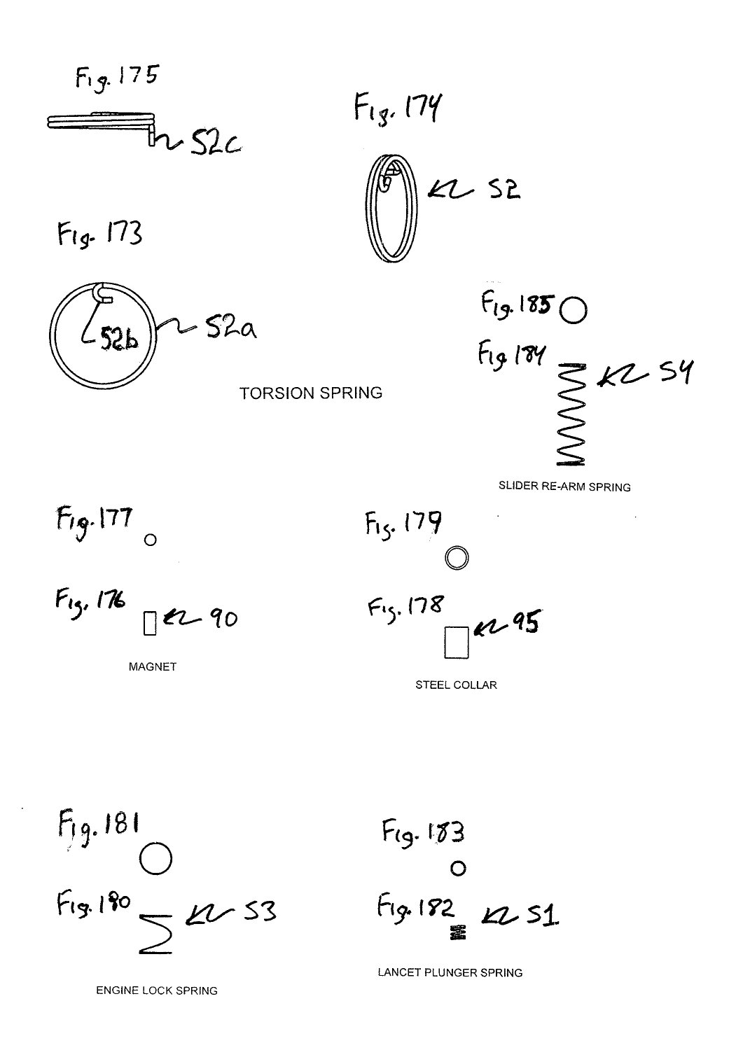

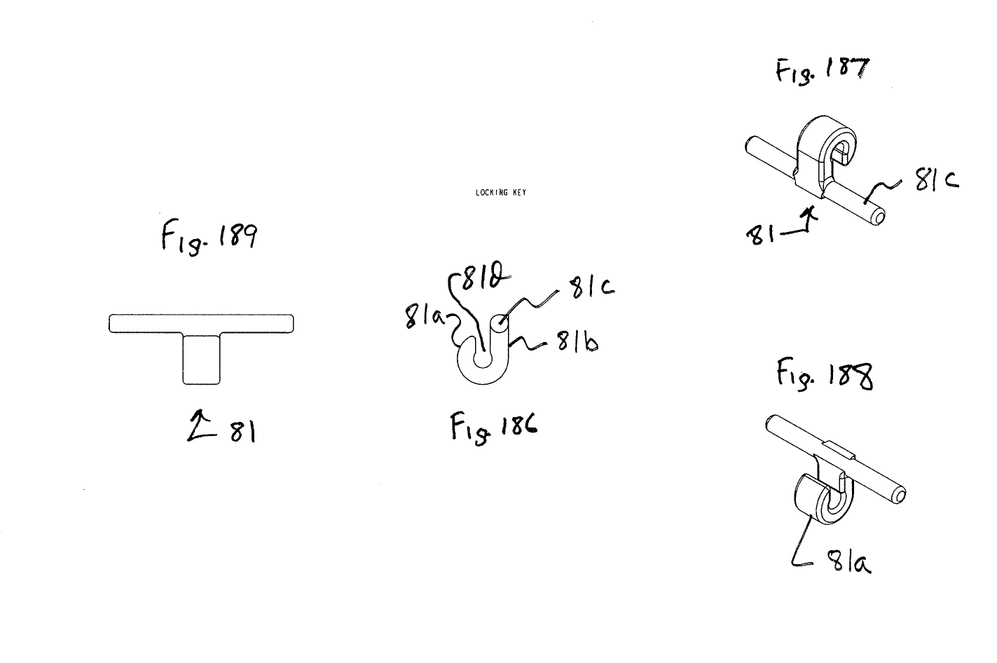

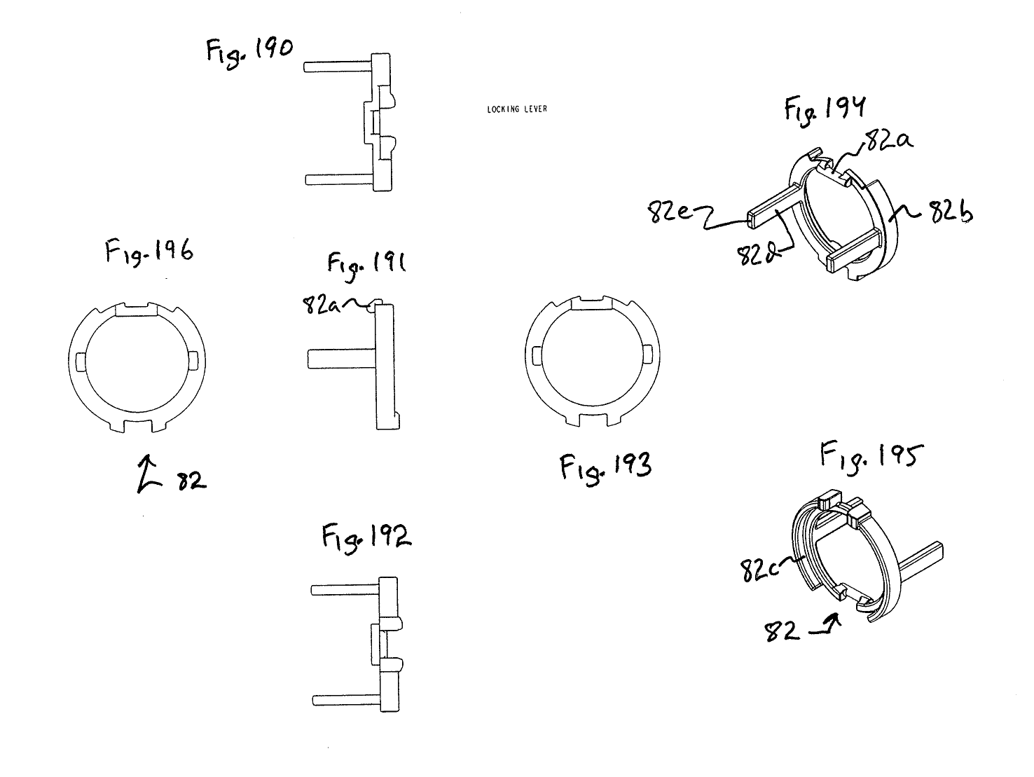

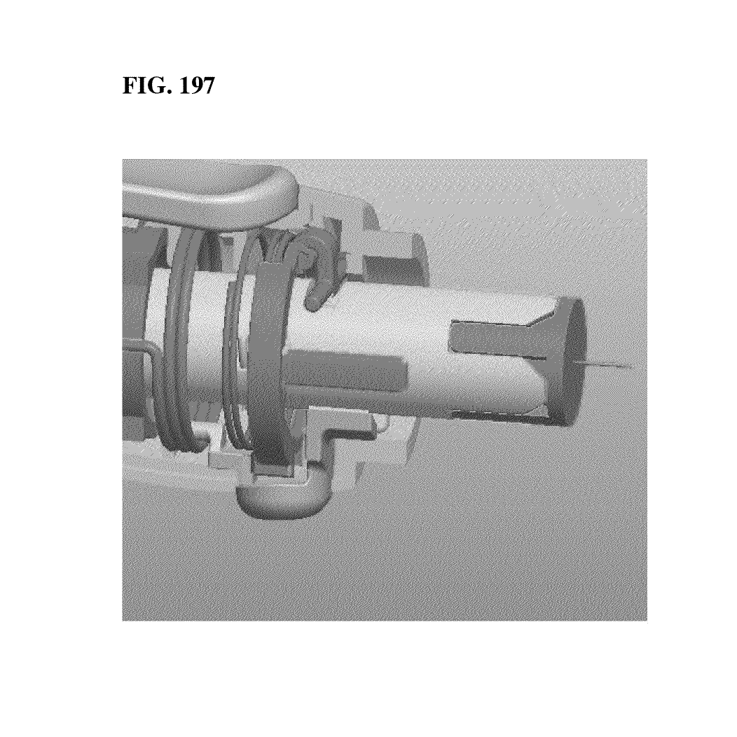

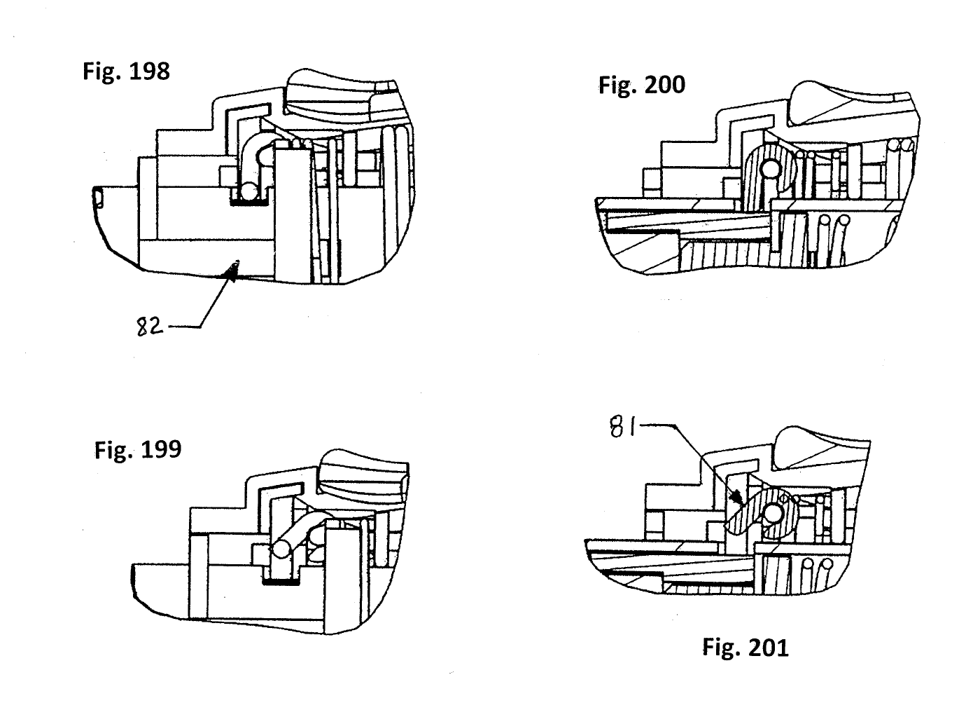

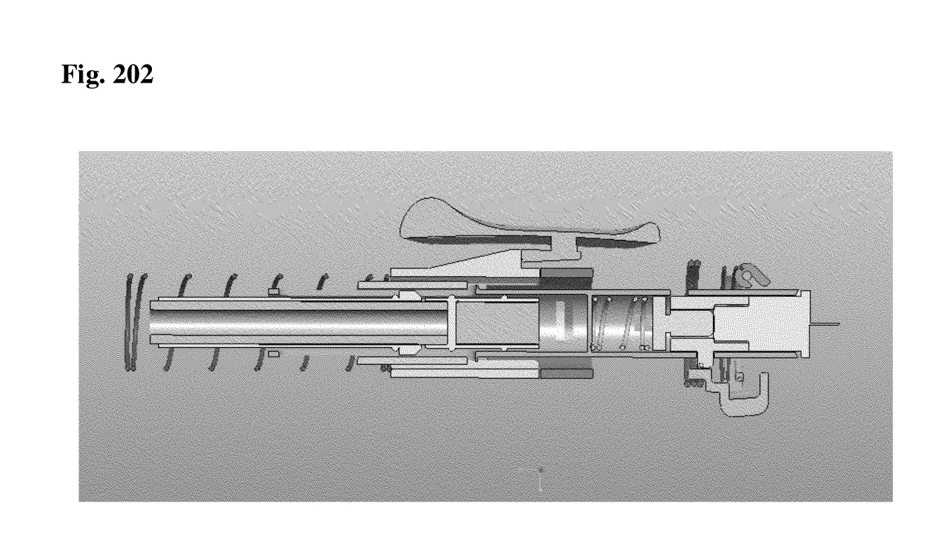

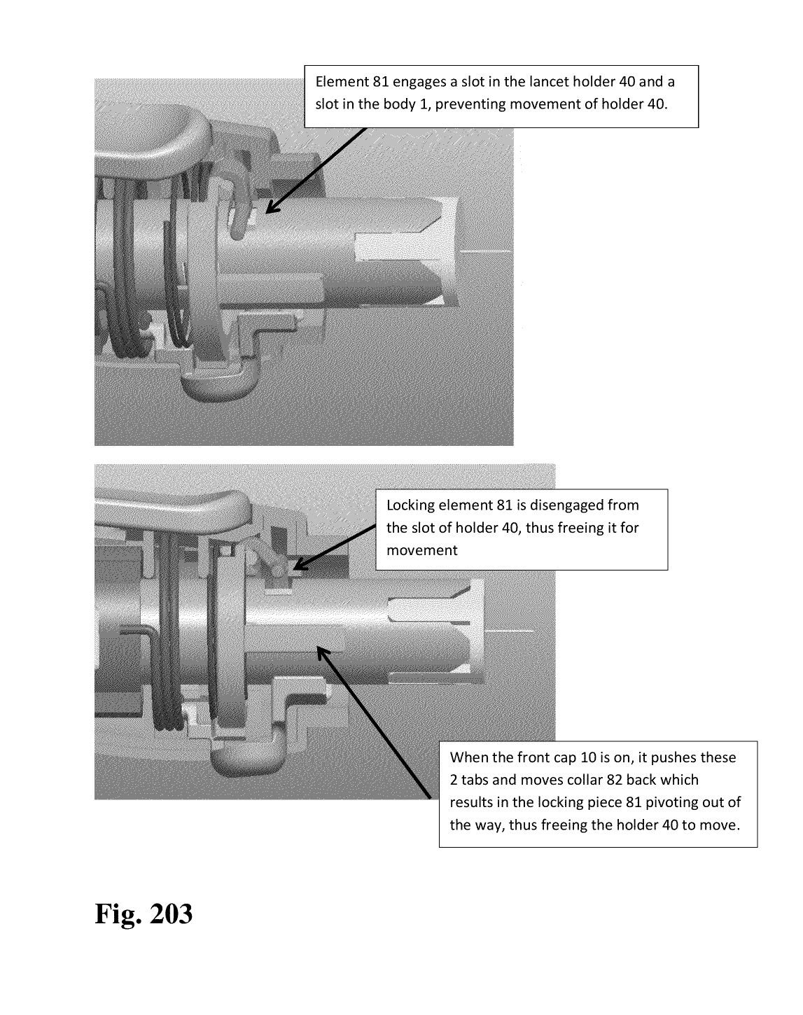

The basic operation of the lancet device LD will now be described with reference to the drawings. Prior to use in forming a puncture, the lancet device LD is made ready. This can typically include installing a lancet L (see FIG. 162) onto the front end of the member 40 (see FIG. 7). To accomplish this, one typically first removes the front cap assembly 10. Doing so automatically results in the member 82 being caused to move forward under the action of the spring S3 (see FIGS. 197-201). This, in turn, causes the lock member 81 to pivot downward so that the end 81c enters into or engages with the slot 40j as shown in FIGS. 197 and 203, and as will be described in detail later on. In this way, the holding member 40 becomes locked in place so that it cannot move axially backwards and forwards. The benefit of this is that the user can now install a lancet L or remove a used lancet (see FIG. 163) and then install a new lancet L. If the latter, the user can rotate member 61 against the biasing force of torsion spring S2 to cause release of the old or user lancet L as shown in FIGS. 164 and 165). Once a new lancet L is installed, the user can reinstall the cap assembly 10. When this happens, the member 82 is caused to move backwards and compresses the spring S3. This, in turn, causes the lock member 81 to pivot upward so that the end 81c exits out of or disengages from the slot 40j and assume the position shown in FIGS. 7, 199 and 201. This results in the holding member 40 being unlocked or freed so that it can now move axially backwards and forwards.

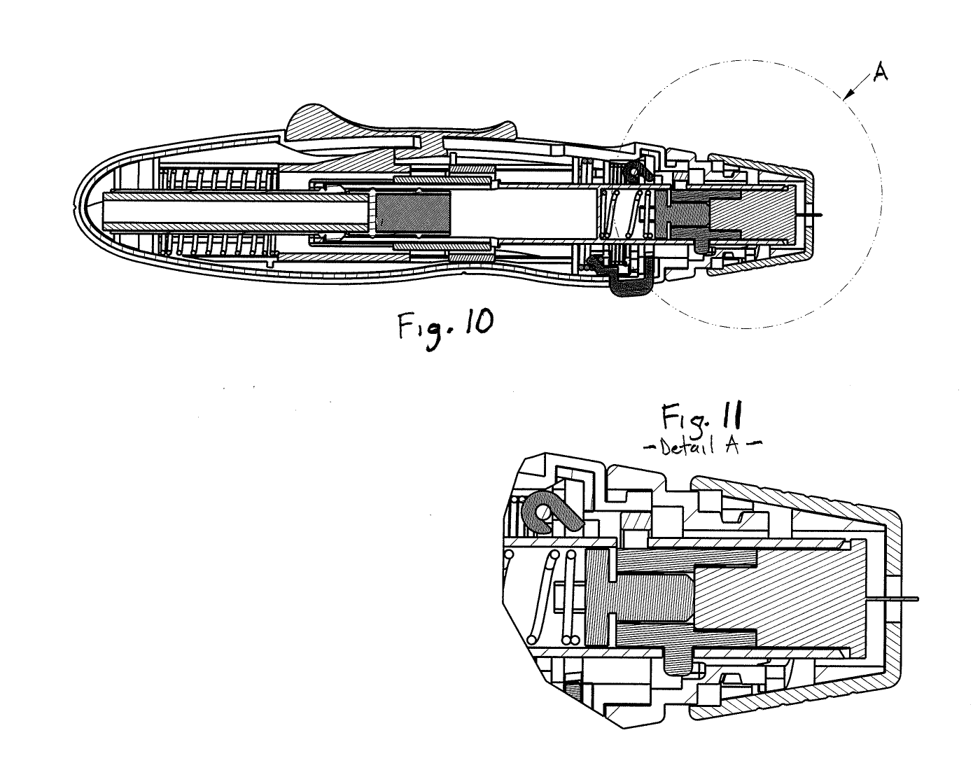

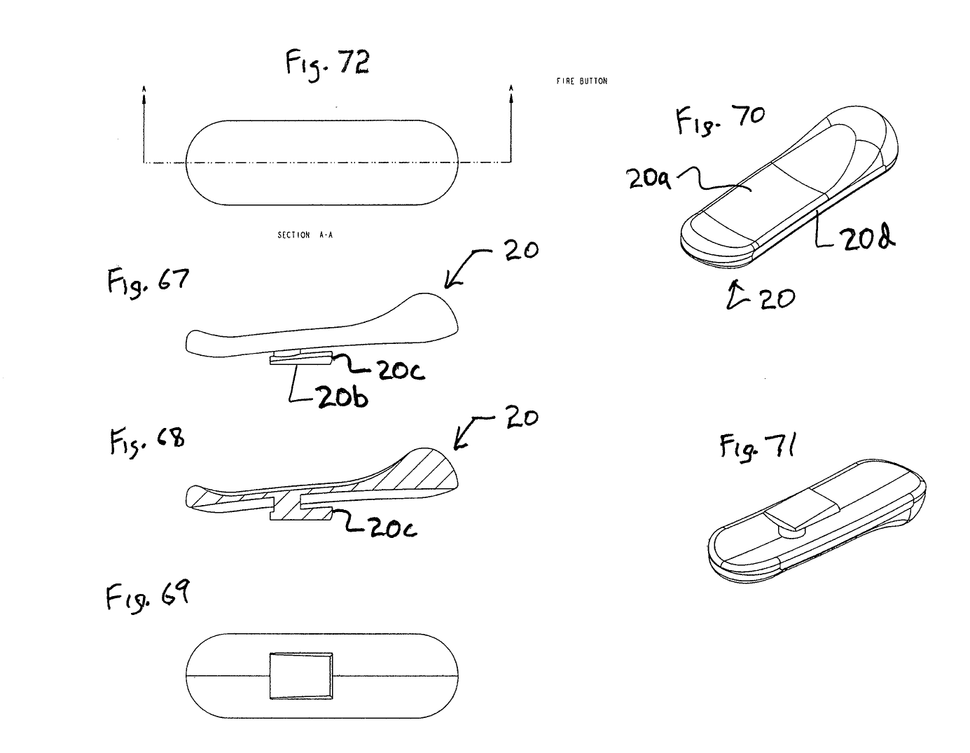

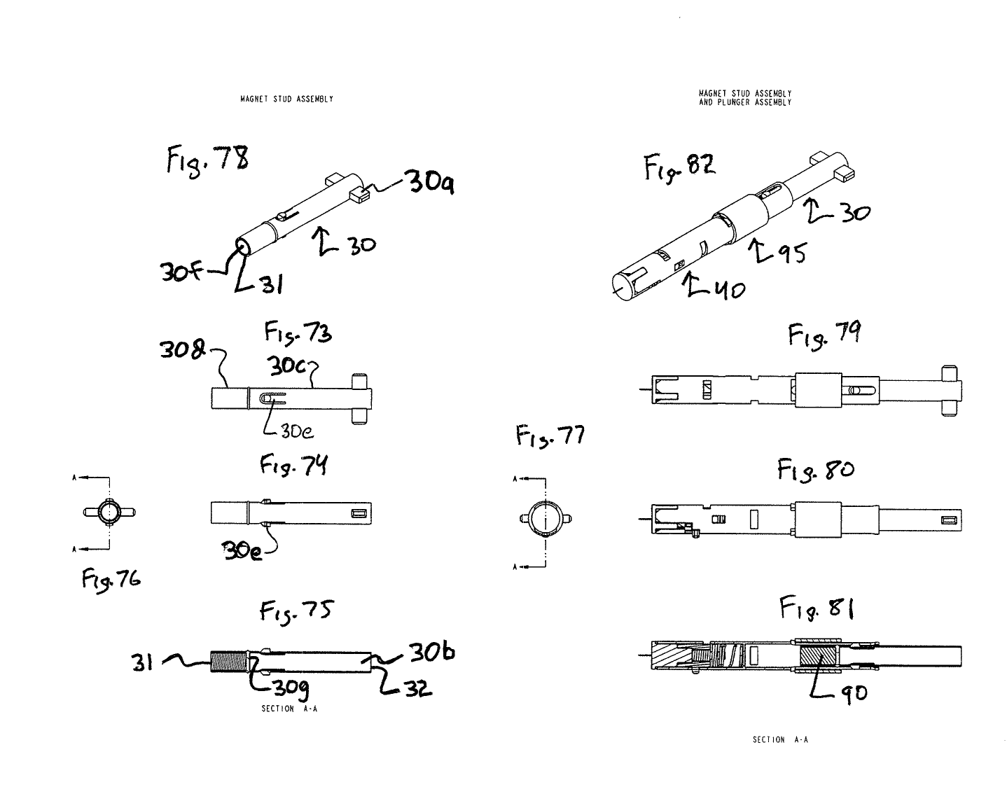

Next, the user can, if desired, set a desired depth of penetration between a minimum and maximum range X as shown in FIG. 41 and also when comparing FIG. 7 and FIG. 10 or 11. This occurs by the user grasping the lancet device LD with one hand while two or more fingers of the other hand grip the cap member 11 and rotate it to a desired setting. When in an intermediate position, rotation of member 11 is one direction will result a shallower puncture while rotation in another direction will result in a deeper puncture. This is because such movement results the skin engaging plane P being positioned to a different axial distance in relation to a maximum axial movement of the holding member 40. Owing to the design and configuration of the magnet 90 and sleeve 95, this maximum movement is repeatedly precisely attained within a relatively small tolerance range. That is, no hard stops are necessary to attain this maximum movement toward the fully extended position unlike many existing lancet devices LD. However, it is also possible to provide for such a hard stop as an additional level of safety and/or as a backup system in case the magnet/sleeve system fail or become compromised for some reason. Maximum movement of the holding member 40 can be limited by the engagement between the slots 40d (see FIG. 83) and the projections 30e (see FIG. 73)—which engagement (see FIG. 82) also prevents rotation of the member 40 while allowing member 40 to move axially relative to both the body 1 and the member 30 which is axially retained in the body 1. An alternative and/or additional stop can be provided by utilizing contact between a proximal surface of the lancet L and the wall of the member 11 containing the lancet opening and plane P. It is desirable, however, that no contact occur between the lancet L and the lancet opening wall as it would render depth adjustment unavailable and generate sounds or noise. The elimination of hard stops is thus similarly desirable. One of many advantageous of using this type of magnetic device is that it is virtually silent. As an alternative depth adjustment procedure, the user can rotate member 11 in relation to member 12 while the cap assembly 10 is removed from the lancet device LD and then install the assembly 10 on the device LD.

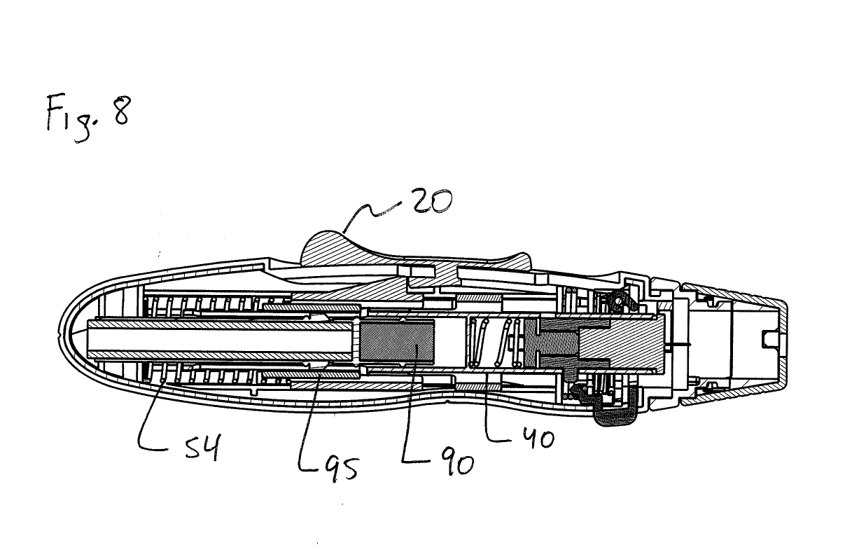

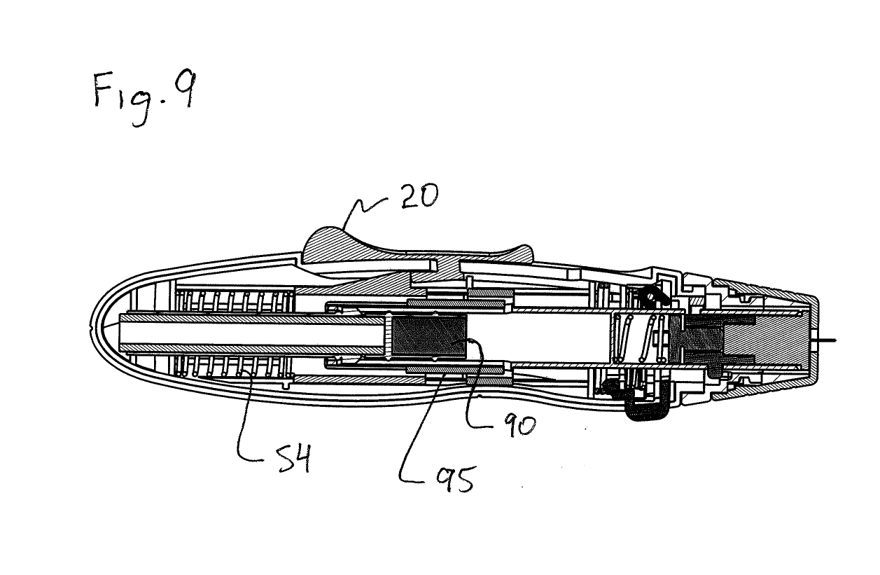

Next, the user can now ready the device LD to form a skin puncture. To do so, the user grasps the lancet device LD with one hand and places the surface P in contact with a skin surface. While still grasping the device LD, the user slide button 20 backwards from the initial position shown in FIG. 7 to that shown in FIG. 8. This action results in axial movement of the members 52 and 51 in a distal direction owing to contact between surface 20c and surface 52i as will be described in detail later on. Furthermore, owing to engagement between projections 52f and slots 40a, the holding assembly 40 is also cause to move axially in a distal direction. This, in turn, results in the sleeve 95 moving from a position centered over the magnet 90 toward a distal direction away from the magnet 90. However, this distal movement toward a retracted position is opposed by the magnet 90 which would quickly bring the sleeve 95 back to the starting position if the slide button 20 were released by the user. This distal movement is also opposed by the spring S4 which becomes compressed by the member 52. During this distal movement, however, the member 52 begins to axially separate from or move away from the member 51. This begins to happen when the projections 51c contact the flanges 2p and 3p which prevent any further distal axial movement of the member 51. As the member 52 is further moved axially in the distal direction by the slide button 20, the sliding engagement between the arms 52e and portions 51f and specifically between surfaces 52g and 51f results in the projection 52f coming out of engagement with the slots 40a. This disengagement can occur generally at the same time as the slide button 20 reaching, or nearly reaching, a distal-most position in the depression 2b/3b as shown in FIG. 9.

As this engagement constitutes the sole mechanism coupling together the holding assembly 40 and the member 52, the holding assembly 40 is now free to move axially to a maximum position shown in FIG. 9 or FIGS. 10 and 11. It is at this precise point that magnet 90 begins to draw the sleeve 95 back to an original position through magnetic force. Of course, because the sleeve 95 is axially fixed to the assembly or member 40, the member 40 with the lancet L arranged at its proximal end is drawn forwardly. The magnetic force exerted by the magnet 90 is such that it causes the sleeve 95 (and member 40) to accelerate in the proximal direction, physics being what it is, the sleeve 95 and member 40 will acquire momentum and not initially stop at a position over the magnet 90. Instead, in a manner analogous to a pendulum, it will travel past the magnet 90 until the force exerted by the magnet 90 overpowers the kinetic energy causing its forward movement—resulting in a deceleration of the member 40 and sleeve 95 to the point where the speed reaches zero. This is the point of maximum travel or the extended position. Furthermore, since the mass and configuration of the sleeve 95 remains constant and because the friction forces resulting from static surfaces contacting the member 40 are similarly constant or essential unchanging, and assuming the magnetic 90 retains its magnetic properties so that the magnetic force remains constant even after many, many uses, the point of maximum travel or the extended position will also be contact or essentially so all other things being equal. After a very short period of time, the sleeve 95 will settle back into a position over the magnet 90 from which it was moved initially. This position is characterized by the magnetic flux emanating from the magnet 90 and entrapping the sleeve 95 so that the forces causing a distal portion of the sleeve 95 to move axially forward are exactly balanced by the forces causing a proximal portion of the sleeve 95 to move axially backward.

Thus, the lancet device LD is both cocked and triggered by distal movement of the slide button 20 between the positions shown in FIGS. 7 and 9. Indeed, it is automatically triggered during cocking of the device LD. Moreover, movement of the holding member 40 toward the extended or puncturing position while the spring S4 remains compressed by the action of the button 20 retaining the movement of the member 52 which has its distal surface in contact with a proximal end of the spring S4. Moreover, because the magnet 90 is fixed within a front end of the member 30 and the member 30 is prevented from moving axially, the magnet 90 remains axially fixed or static throughout all movements of the button 20, members 51 and 52, and especially member 40 and sleeve 95.

Once the skin is punctured by the needle N of the lancet L passing through the lancet opening LO, the user can release the button 20 which will automatically be brought or moved forward via the member 52 under the action of the spring S4. During this movement, the member 51 is ultimately cause to contact the wall 2k/3k and is stopped. However, the member 52 (which never completely separated from member 51) continues to move forward until the projections 52f are allowed to deflect inwardly and again engage with the slots 40a of the member 40. At this point the device LD is back to an original position as shown in FIG. 7 and is ready to be used for another skin puncture—although it is recommended that the lancet L be replaced with a new one before this occurs.

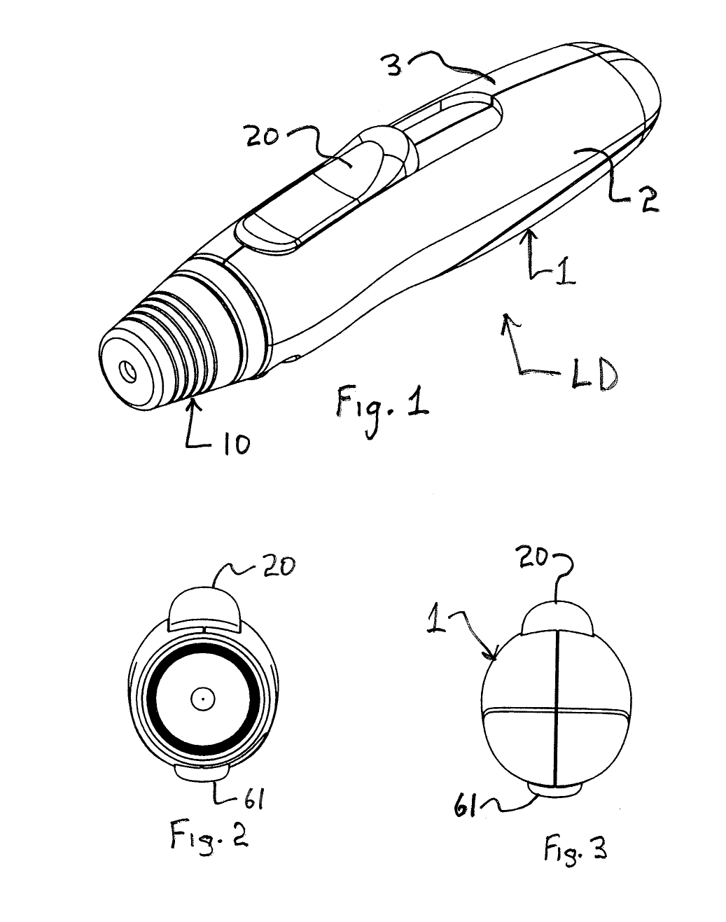

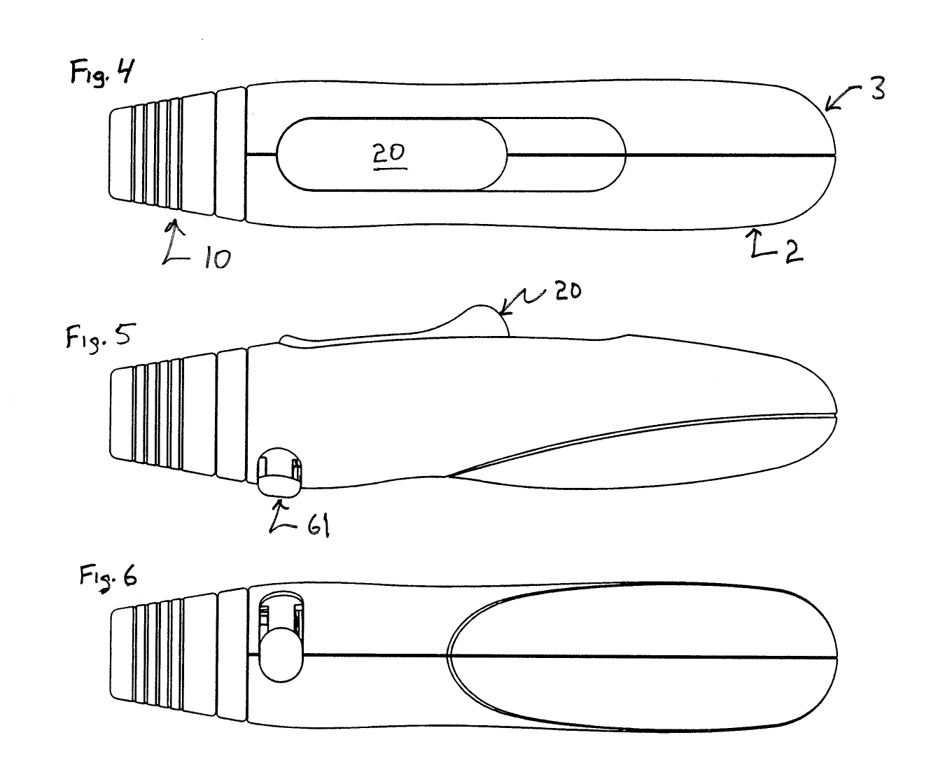

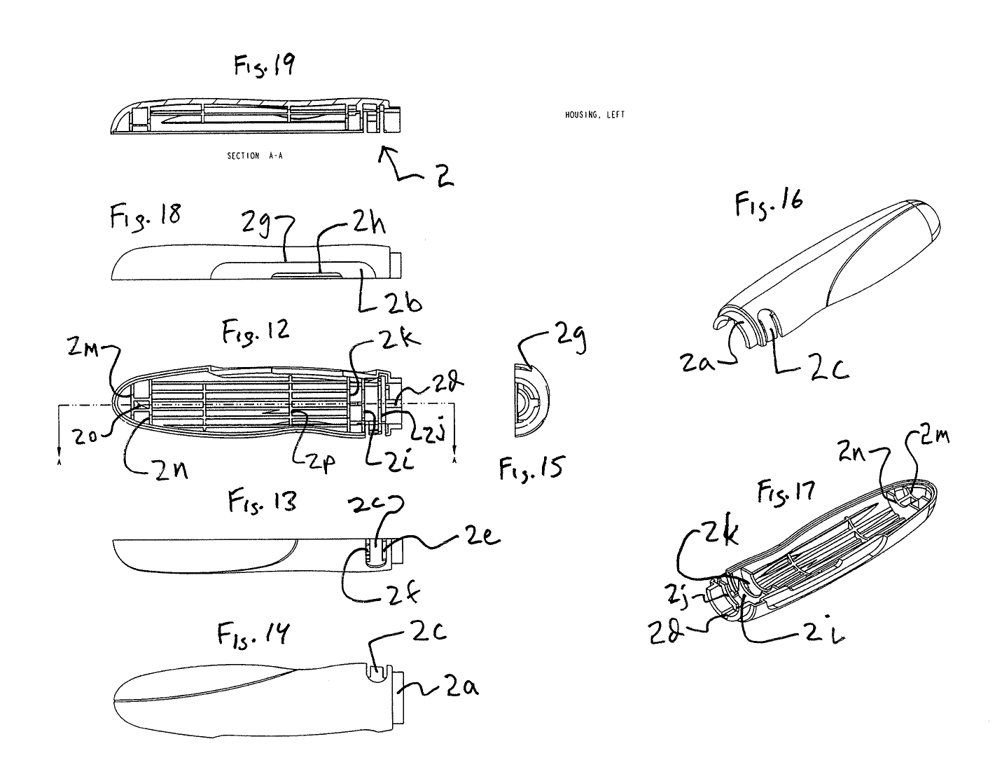

With reference to FIGS. 12-27 it can be seen that the body 1, in accordance with one non-limiting embodiment, utilizes main components 2 and 3 and has an open front end and a closed and rounded rear end. The shape of the body 1 is generally made ergonomic (see FIGS. 1-6) so that a user can more comfortably use and handle the same. This also makes it less effortless to activate or move the slide button 20 and release button 61. A first component is a left-side body section or first portion 2 of the body 1. A second component is a right-side body section or second portion 3 of the body 1. When body sections 2 and 3 are joined together, they form a generally cylindrical connecting section via partially curved portions 2a and 3a. These sections 2a and 3a form a generally cylindrical surface which can receive thereon the surface 12d of the cap assembly 10 (see FIGS. 16 and 24). Also formed is a recess or depression defined by portions 2b and 3b. These are sized and configured to receive therein the slide button 20. Additionally, there is also formed is a recess or depression defined by portions 2c and 3c. These are sized and configured to receive therein the release button 61.

Body section 2 also includes a guide depression 2d sized and configured to receive therein and linearly guide one of the arms 82d of the member 82 (see FIGS. 190-196). Body section 3 similarly also includes a guide depression 3d sized and configured to receive therein and linearly guide another of the arms 82d of the member 82. Body section 2 includes two through slots 2e and 2f sized and configured to receive therein and linearly guide portions 61c and 61e of the member 61 (see FIGS. 166-172). Side surface 2g is sized and configured to linearly guide the slide button 20 and can be contacted or slidably engaged by surface 20d (see FIGS. 67-72). Similarly, side surface 3g is sized and configured to linearly guide the slide button 20 and can be contacted or slidably engaged by surface 20d. An elongated through opening or slot is formed by portions 2h and 3h which together are sized and configured to receive therein portion 20b of the slide button 20. A generally circular flange is formed by portions 2j and 3j which together are sized and configured to form a front wall. Another generally circular flange is formed by portions 2i and 3i which together are sized and configured to form a distal wall. A space formed between these walls is sized and configured to receive therein the spring S3 and member 82. Another generally circular flange is formed by portions 2k and 3k which together are sized and configured to form a front wall. A space formed between the walls 2i/3i and 2k/3k is sized and configured to receive therein the spring S2 and portion 61f. Notch 31 formed in section 3 is sized and configured to receive therein and retain end S2c of the spring S2.

Body section 2 also includes flanges 2m and 2n and well as a generally rectangular opening 2o which is sized and configured to receive therein and axially and rotatably retain one of the projections 3a of member 30. Similarly, body section 3 includes flanges 3m and 3n and well as a generally rectangular opening 3o which is sized and configured to receive therein and axially and rotatably retain another of the projections 3a of member 30. The flanges 2n and 3n also serve as contact surface for the distal end of the spring S4.

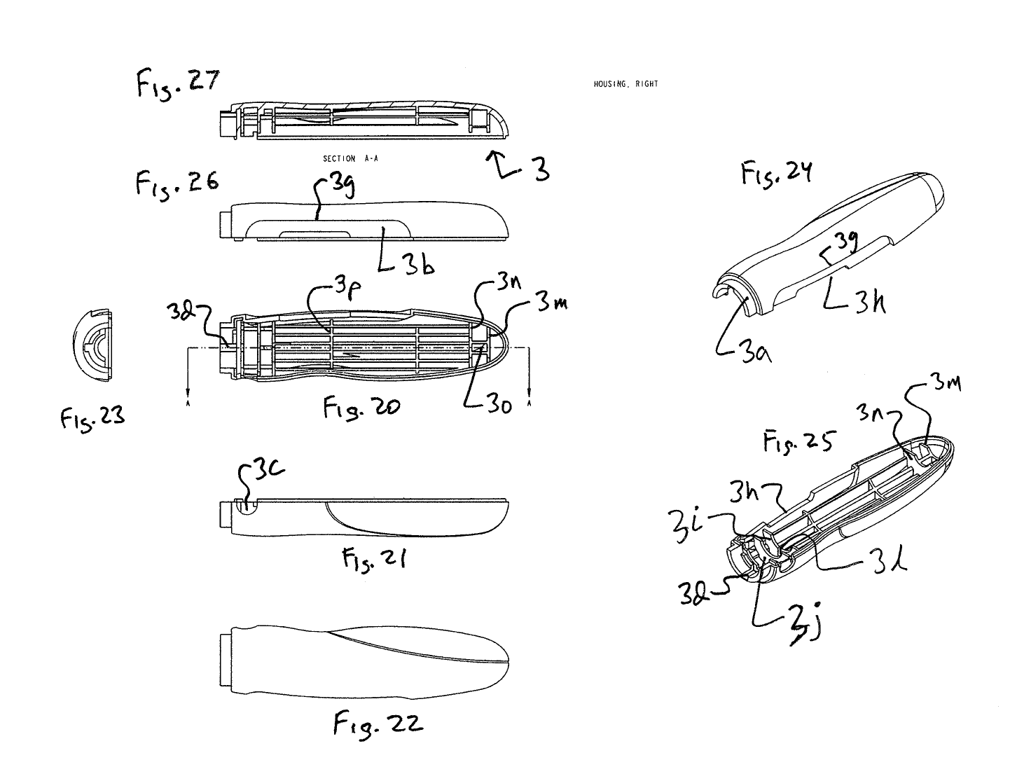

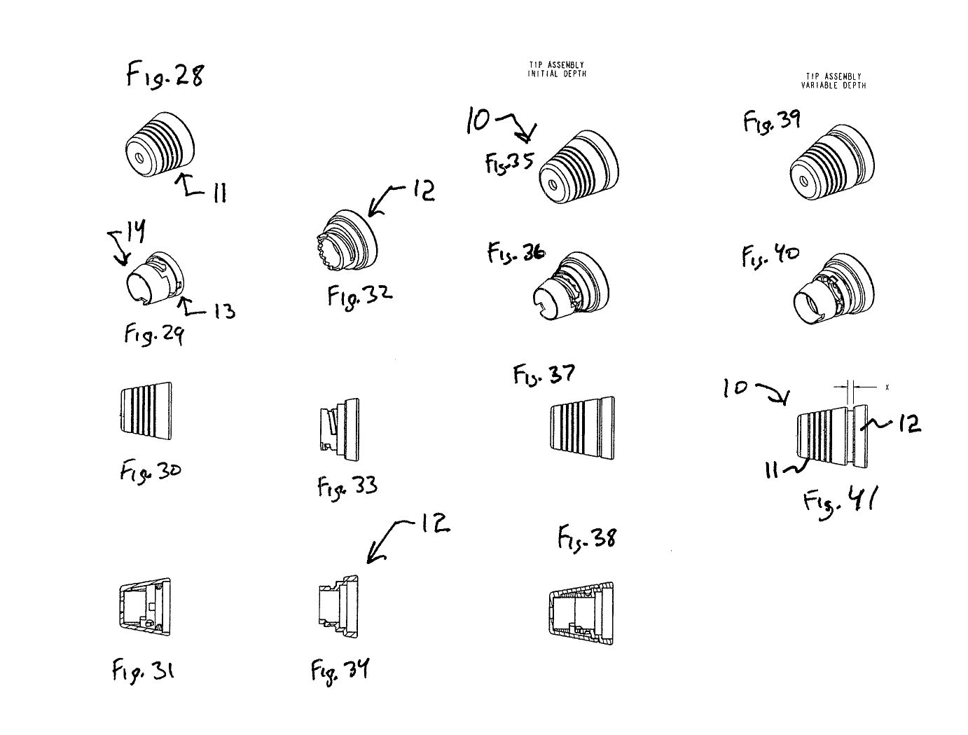

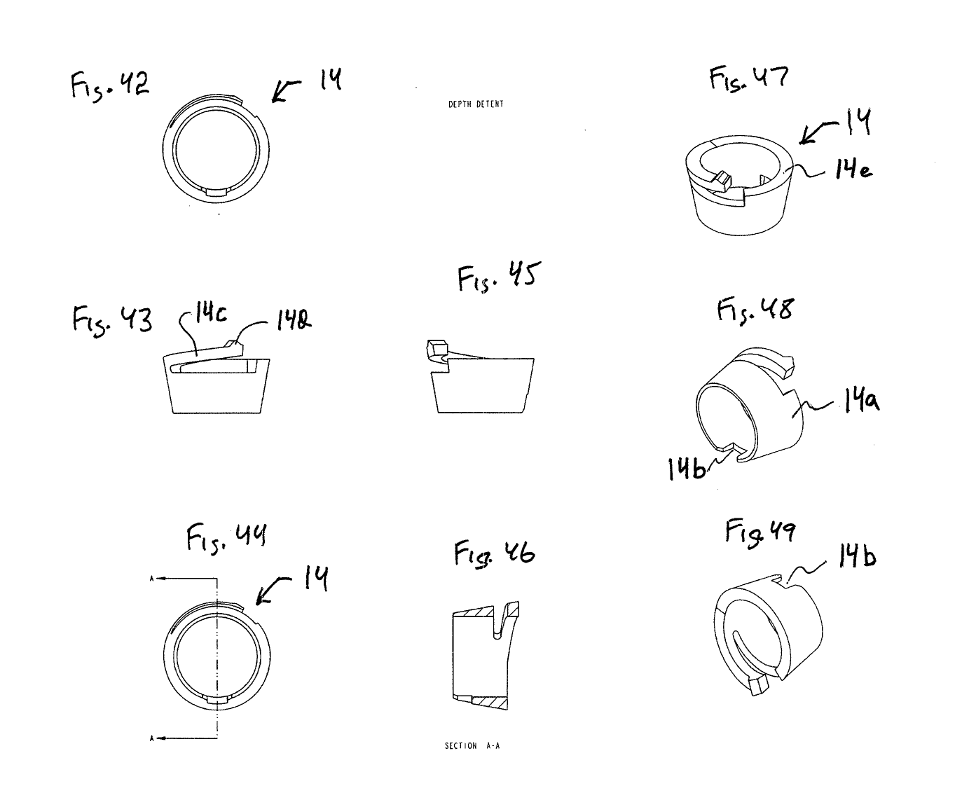

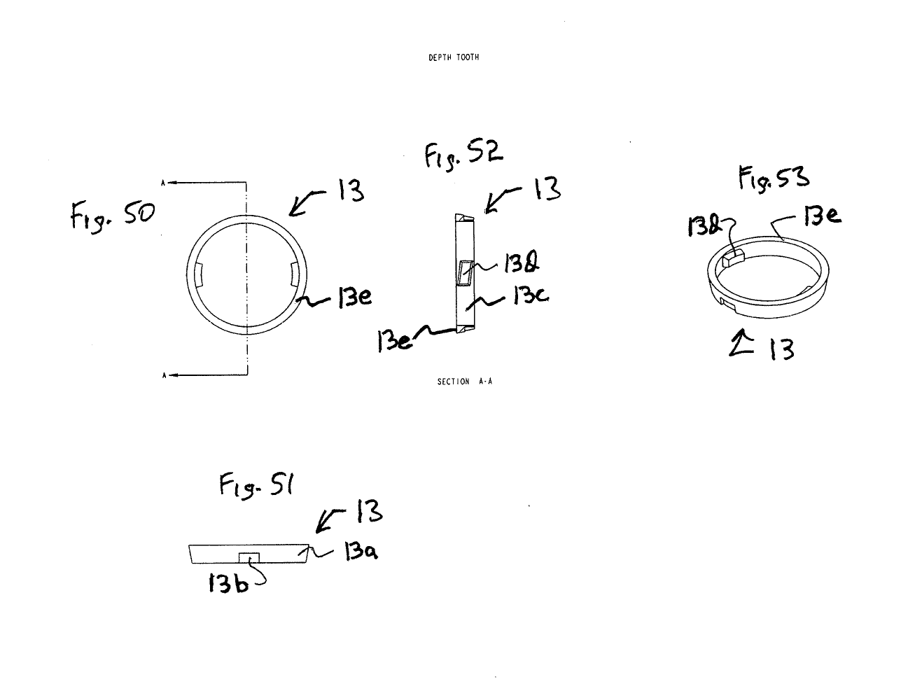

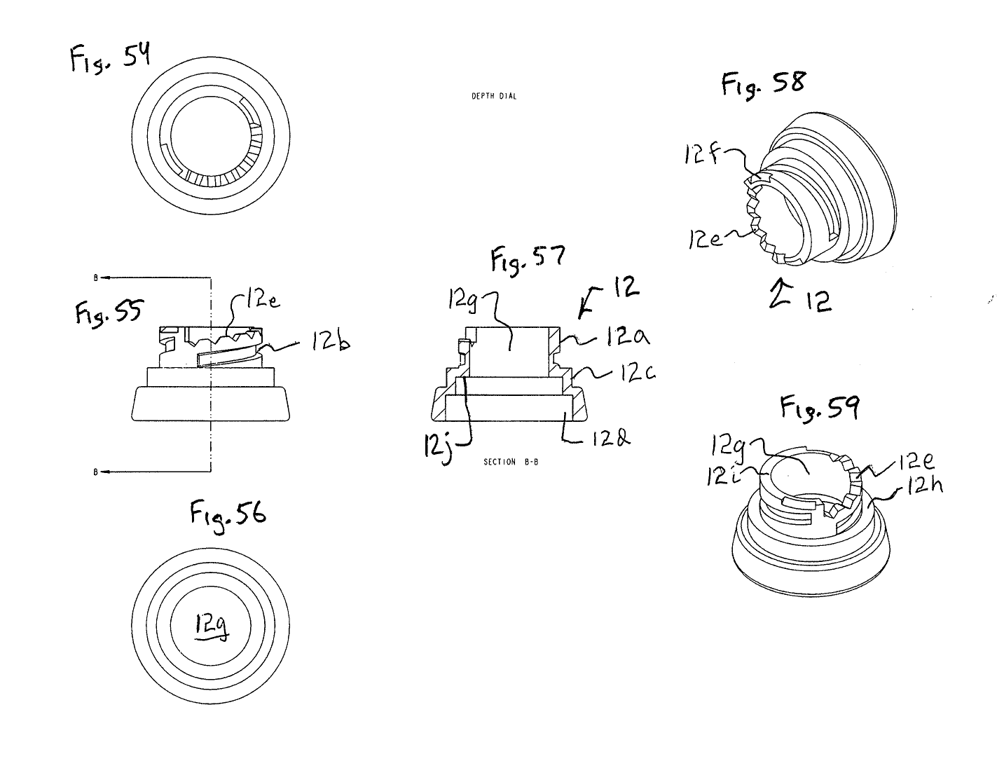

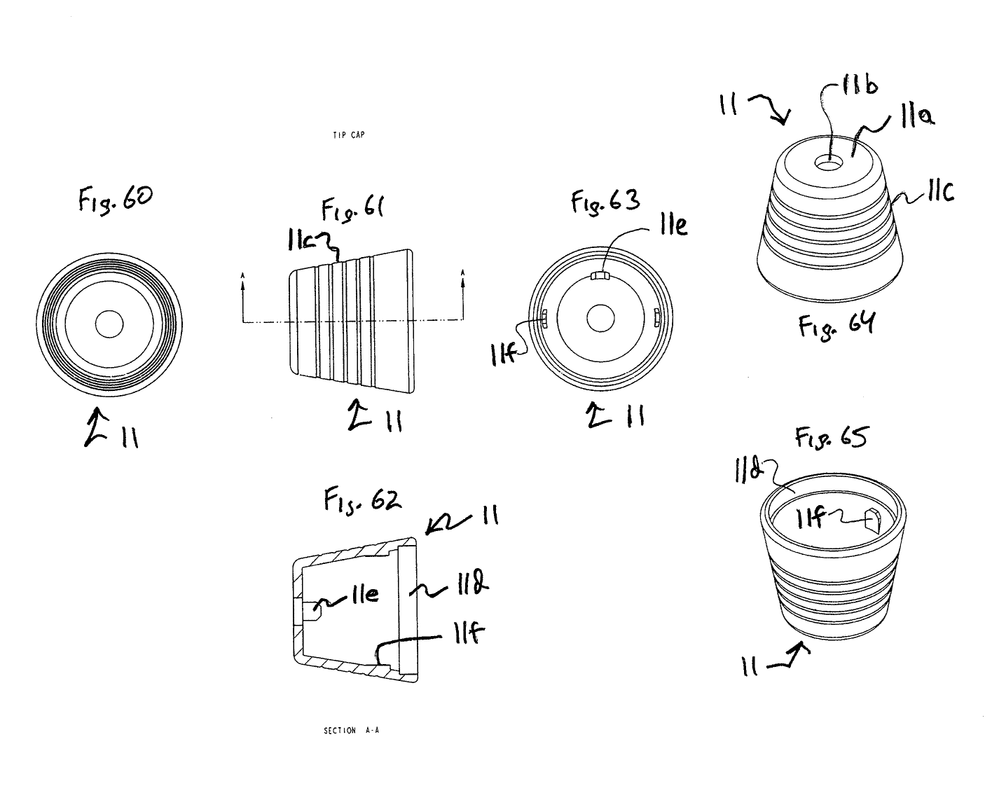

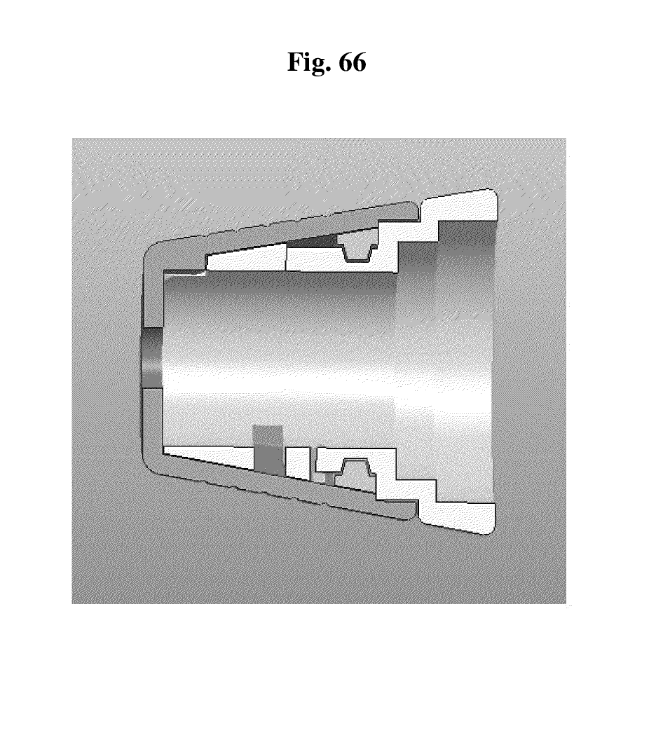

With reference to FIGS. 28-66, it can be seen that the cap assembly 10, in accordance with one non-limiting embodiment, utilizes main components 11-14. A first component is a cap member or cap portion 11 of the assembly 10. A second component is a distal member or connecting interface portion 12 of the assembly 10. A third component is a ring member or ring portion 13 of the assembly 10. A fourth component is a retaining member or retaining portion 14 of the assembly 10.

The cap member 11 includes a skin engaging surface 11a and a lancet opening 11b through which a lancet needle can pass during puncturing of the skin or tissue. Surface 11a can be generally planar as shown or alternatively can be inwardly or outwardly curved as is utilized n known lancet devices. This surface can also include dimples or projections which are also known in lancet devices in order to, among other things, provide skin stimulation. Opening 11b can be generally circular as shown or alternatively can have another shape. Additionally, this opening can be of any desire size although it can be desirable that this opening not be too large so as to allow the skin surface to extend too deep into the opening and thereby allow too deep a skin puncture.

Cap member 11 also includes an outer surface 11c which can in this case be generally tapered. Surface 11c is sized and configured to be gripped by a user so as to allow the user to rotate the same in relation to component 12 which will be described in detail below. This rotation can be in opposite directions to provide depth adjustment and is facilitated by the surface 11c having a high friction area which in this case can take the form of grooves and projections that are more easily gripped that a plain and/or a more slippery surface. Other friction increasing devices can also be utilized such as a knurl or other type of textured surface. Cap member 11 also includes, at a distal end, a generally circumferential engaging surface 11d. This surface 11d is sized and configured to rotatably and/or axially slidably engage with surface 12c of component 12. An internal key projection 11e is arranged within the cap 11 and is sized and configured to engage with slot or recess 14b of component 14. When so engaged, rotation of the cap 11 will cause a corresponding rotation of component 14. Cap member 11 further also includes two generally oppositely arranged engageing projections 11f. These projections 11f are sized and configured to lockingly engage with corresponding recesses 13b of component 13. This serves to retain the cap 11 on the assembly 10.

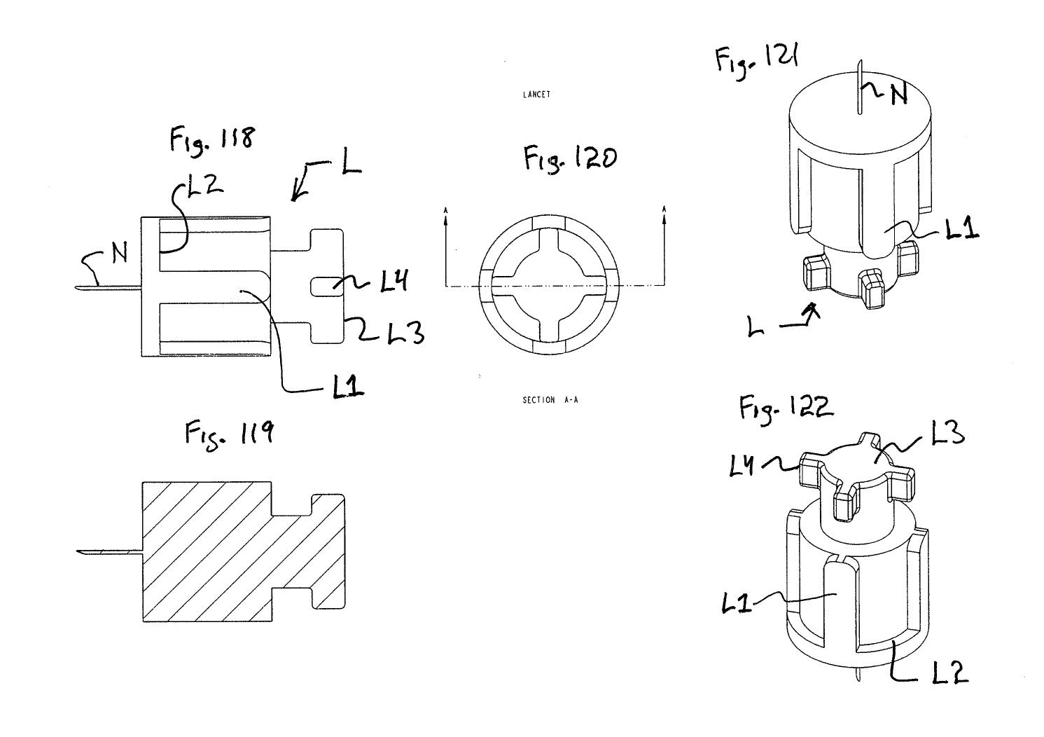

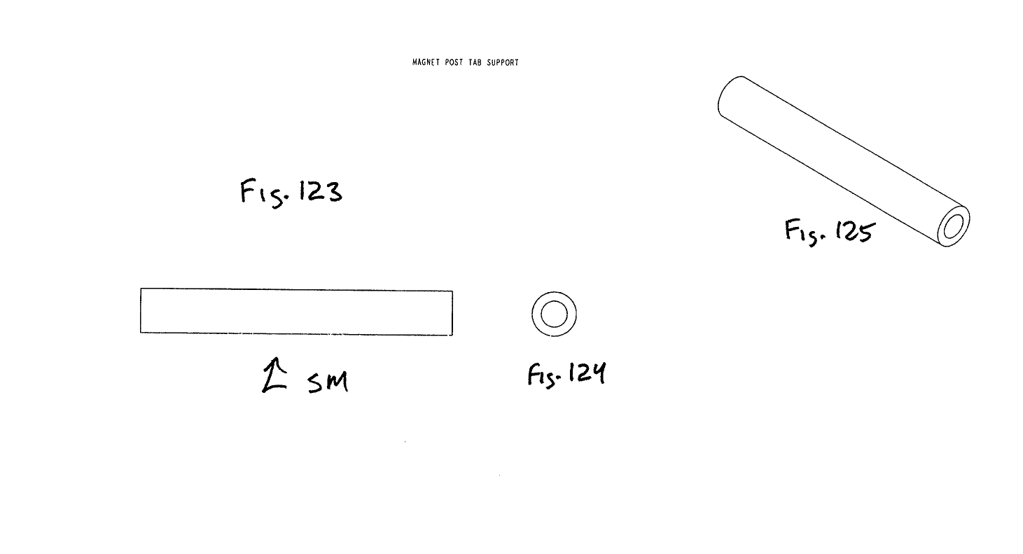

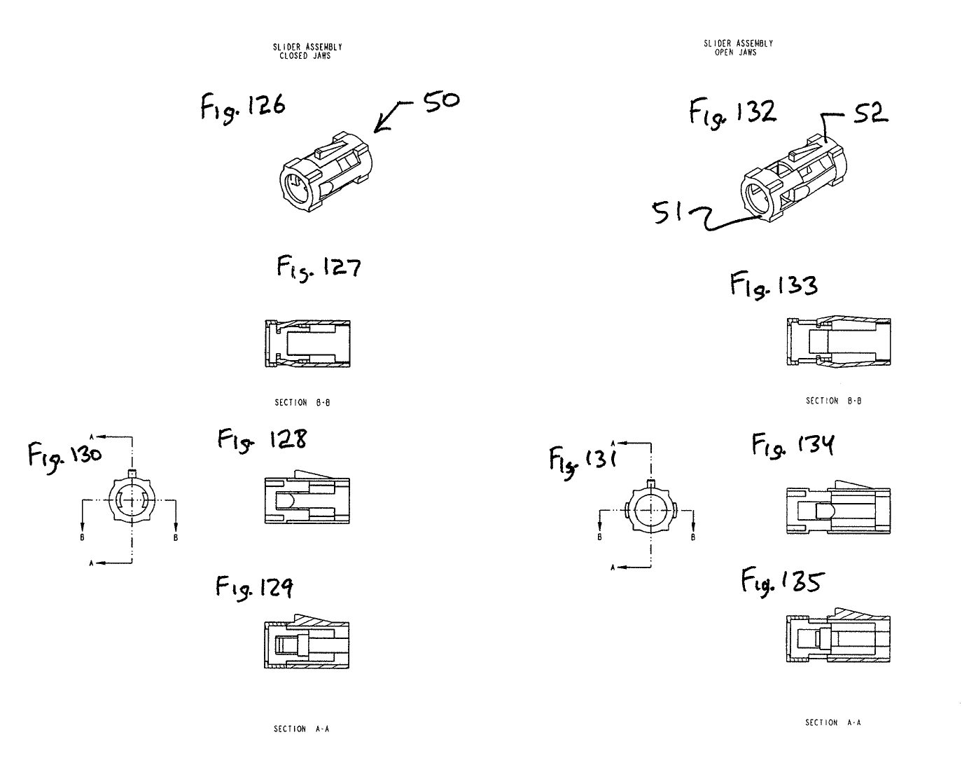

Component 12 includes an outer surface 12a which can in this case be generally cylindrical. Surface 12a is sized and configured to slidably or rotably engage with a comparable inside surface 13c of component 13. A helical slot 12b is sized and configured to receive therein two generally oppositely arranged inwardly oriented projections 13d of the member 13. Due to this engagement, when the cap 11 and ring 13 are rotated, the projections 13d move in and are guided by the slot 12b in such a way that the cap 11 can both rotate relative to member 12 and also be guided axially relative thereto. As a result of this engagement, when the cap 11 is rotated in one direction, the cap 11 moves axially toward the member 12 whereas when the cap 11 is rotated in an opposite direction, the cap 11 moves axially away from the member 12. This adjustment movement regulates the position of the skin engaging surface 11a in relation to a non-changing maximum position of the lancet needle N (see FIGS. 118-122) such that when the cap 11 is rotated in one direction, the cap 11 moves axially toward the member 12 and thereby provides a deeper skin puncture than when the cap 11 is rotated in an opposite direction, with the cap 11 moving axially away from the member 12.

Generally cylindrical surface 12c is sized and configured to be rotatably and/or axially slidably engaged by surface 11d of component 11. Generally cylindrical surface 12d is sized and configured to be mounted to a corresponding surface of the body 1 of lancet device LD as will be described below and, in particular, to surfaces 2a and 3a of body sections 2 and 3. This mounting forms a connection between the cap assembly 10 and the body 1. Arranged at a proximal end of the component 12 are a series of projections and recesses 12e which provide distinct retaining locations for a deflectable projection 14d of the component 14. The recesses 12e differ from one another in circumferential and axial spacial location and, in cooperation with the slot 12b and projections 13d of component 13 serve to provide different depth of penetration settings. By rotating the cap 11 in relation to the lancet device LD and/or the component 12, the user causes the projection 14d to move into and out of engagement with a different one of the recesses 12e. Moreover, due to this arrangement, an audible clicking “click” sound is generated each time the depth setting is changed. Additionally, the component 12 includes tapered indentations 12f and a through main opening 12g.

Component 13 includes an outer surface 13a which can in this case be generally tapered. Surface 13a is sized and configured to frictionally engage with a comparable inside surface of component 11 and includes recesses 13b sized and configured to receive therein two generally oppositely arranged inwardly oriented projections 11f of the member 11. In embodiments this surface 13a is non-removably or permanently fixed, via e.g., adhesive bonding or ultrasonic welding, to the comparable inside surface of the cap 11. Component 13 also includes projections 13d as described above and an annular surface 13e sized and configured to rotatbly engage with comparable annular surface 12h of the member 12.

Component 14 includes an outer surface 14a which can in this case be generally tapered. Surface 14a is sized and configured to non-movably engage with a comparable inside surface of component 11 and includes a recess 14b sized and configured to receive therein the projection 11e of the member 11. As a result, any rotation movement imparted to the cap 11 is transferred to the component 14 so that it rotates with the cap 11. A deflectable arm 14c is sized and configured to exert an axial biasing force against component 12. This biasing force ensures that the projection 14d clicks into and out of engagement with the recesses 12e of component 12 when the cap 11 is rotated. The annular surface 14e rotatably engages with annular surface 12i of the member 12.

Referring now to FIGS. 67-72 it can be seen how the slide or movable/slidable arming and trigger button 20, in accordance with one non-limiting embodiment, can be a one-piece or integrally formed member. The slide 20 includes a finger engaging surface 20a which is sized and configured to comfortably receive a thumb when the lancet device LD is properly grasped by a user. Two oppositely arranged surfaces 20d slidably engage with surfaces 2g/3g of the body 1 and this engagement serves to linearly guide the slide 20. The slide 20 also includes a projecting portion 20b sized and configured to extend within the body 1. The projection 20b has a distal projecting surface 20c which is configured to contact surface 52i of the projection 52h.

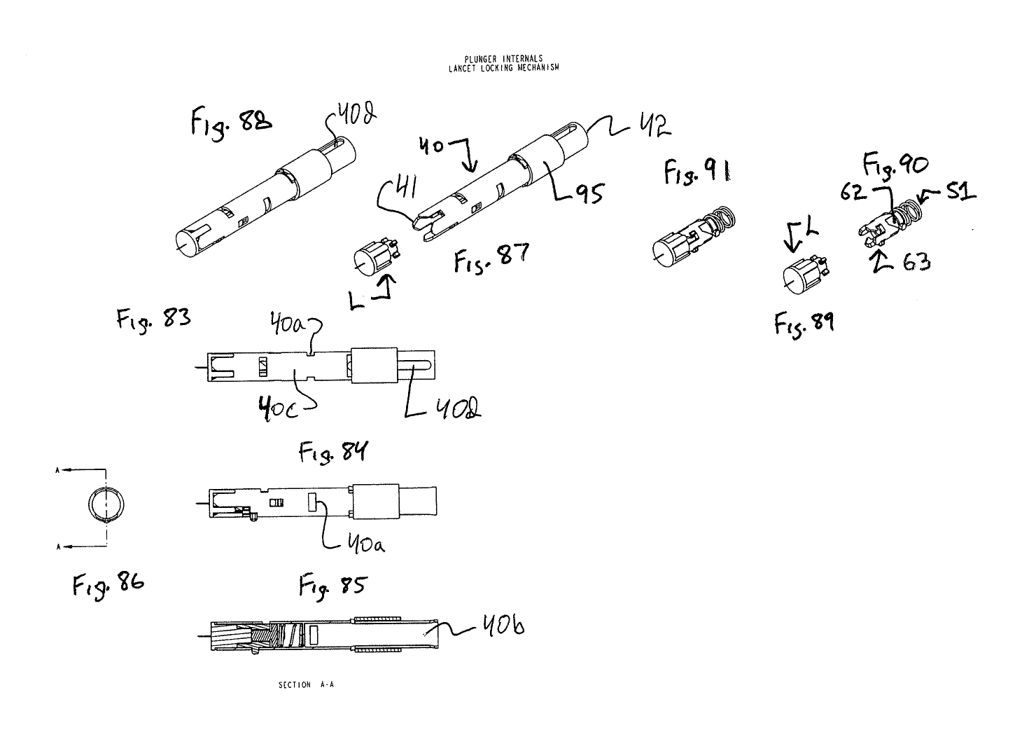

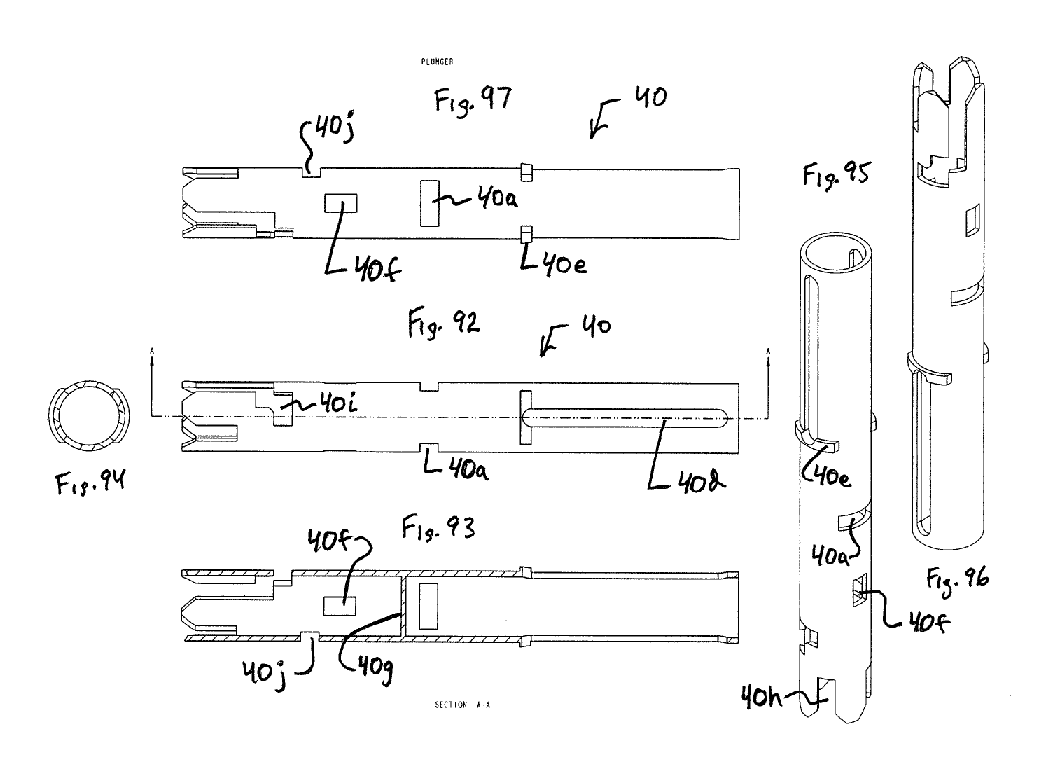

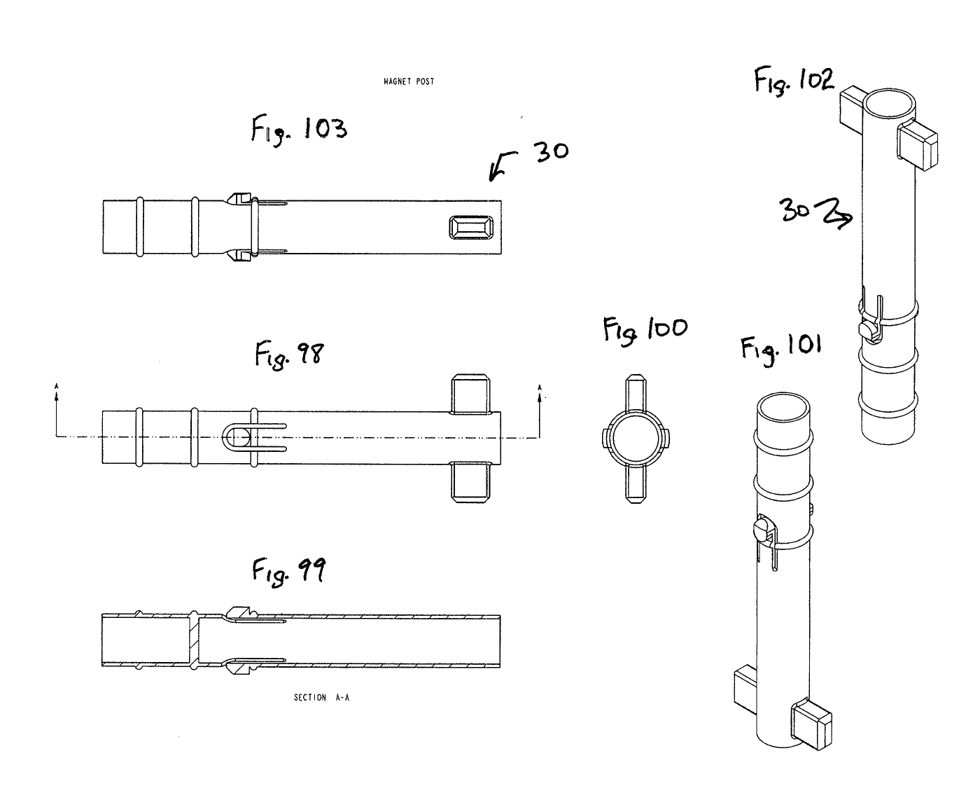

Referring to FIGS. 73-104, it can be seen that the lancet holder or holding assembly, in accordance with one non-limiting embodiment, utilizes main components 30 and 40. A first component is a proximal member or front portion 40 of the assembly. A second component is a distal member or rear portion 30 of the assembly. A metal sleeve 95 is slid over and is axially retained on a generally central area of the assembly. The sleeve 95 is that part of the lancet device LD which is influenced by a magnet 90, and owing to its fixed connection to the holder 40, causes axial movement of the holder assembly 40.

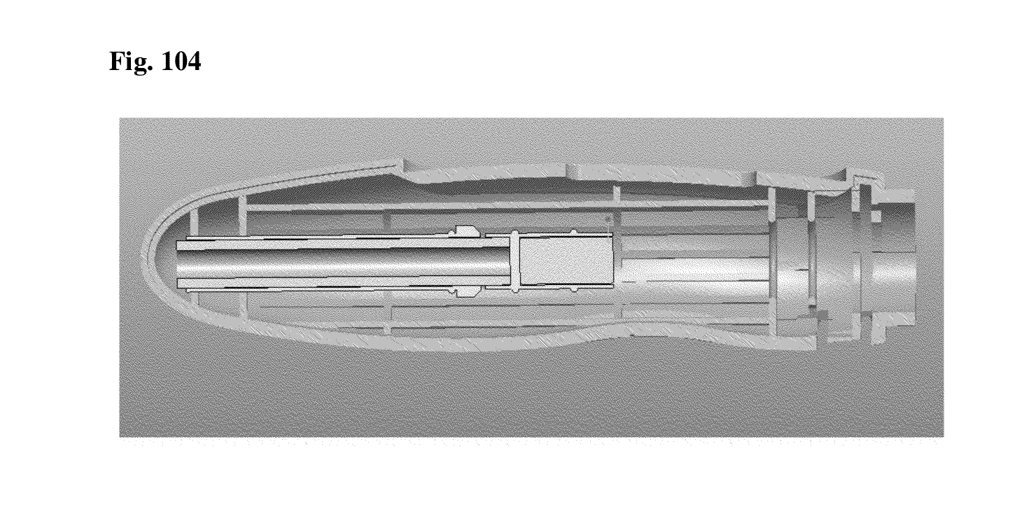

The distal member 30 has a first end 31 and a second end 32 and includes two oppositely arranged projections 30a which are sized and configured to be axially retained between two flange portions 2n/3n and 3n/3m of the body 1 (see FIGS. 12-27 and 104). A generally cylindrical internal space 30b is disposed within the member 30. In embodiments, the space 30b is sized and configured to receive therein a spacer member SM (see FIG. 104). A generally cylindrical outer surface 30c of the member 30 is sized and configured to at least partially extend into the distal end of a generally cylindrical open space 40b of the member 40. Once a proximal end 30d of the member 30 is inserted into a distal end of the member 40, two oppositely arranged deflectable projections 30e are sized and configured to slide within a respective slot of two oppositely arranged slots 40d of the member 40. Even though the member 30 is axially fixed within the body 1, the member 40 can still move axially back and forth. The proximal end 30d of the member 30 includes a proximal generally cylindrical space 30f which is sized and configured to frictionally and/or fixedly receive therein the magnet 90. Assembly of the member 30 with the member 40 can be accomplished by first installing the magnet 90 within the space 30f until the magnet 90 abuts an internal wall 30g of the member 30 and installing the metal sleeve 95 onto the member 40 until the metal sleeve 95 abuts the outer projections 40e of the member 40. After this occurs, one can insert the proximal end 30d into the member 40 by an amount sufficient to ensure that the projections 30e become engaged with the slots 40d of the member 40.

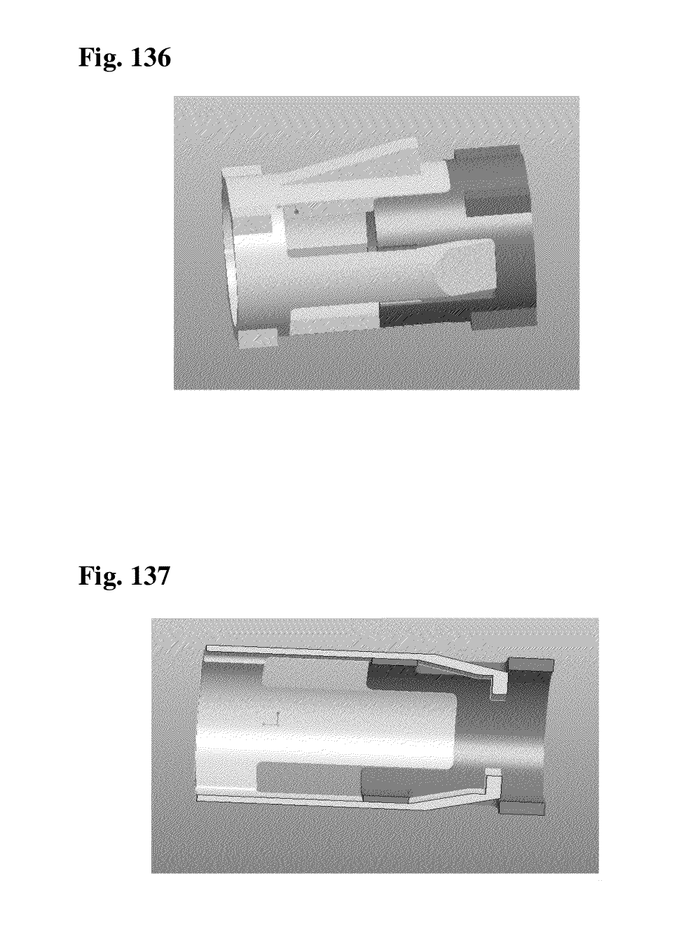

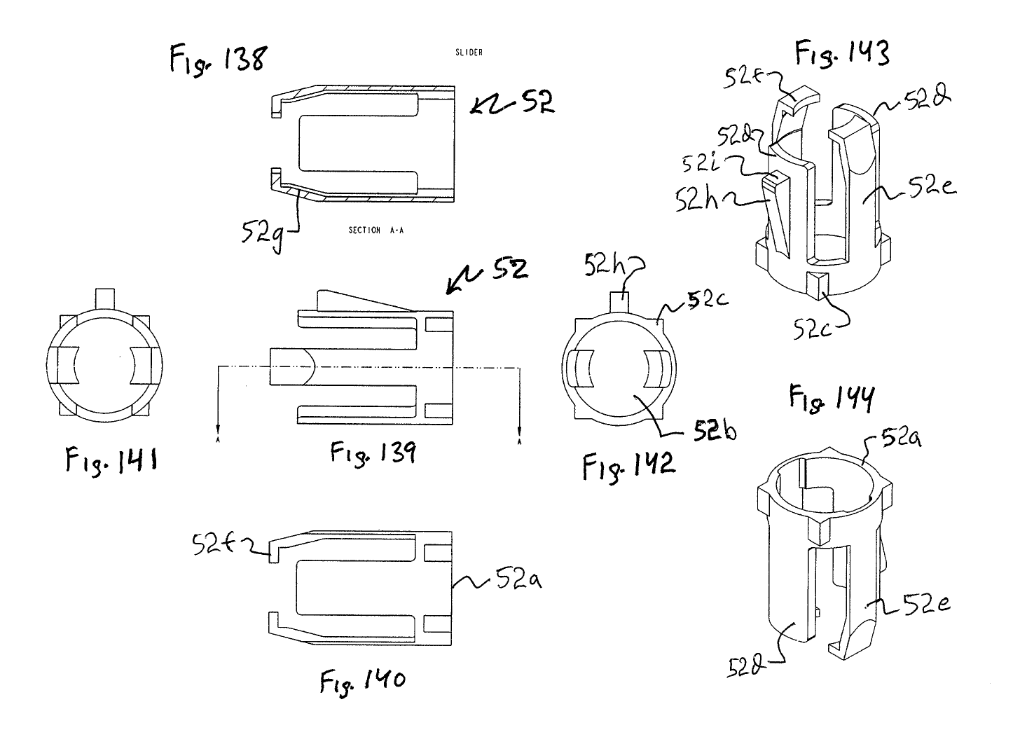

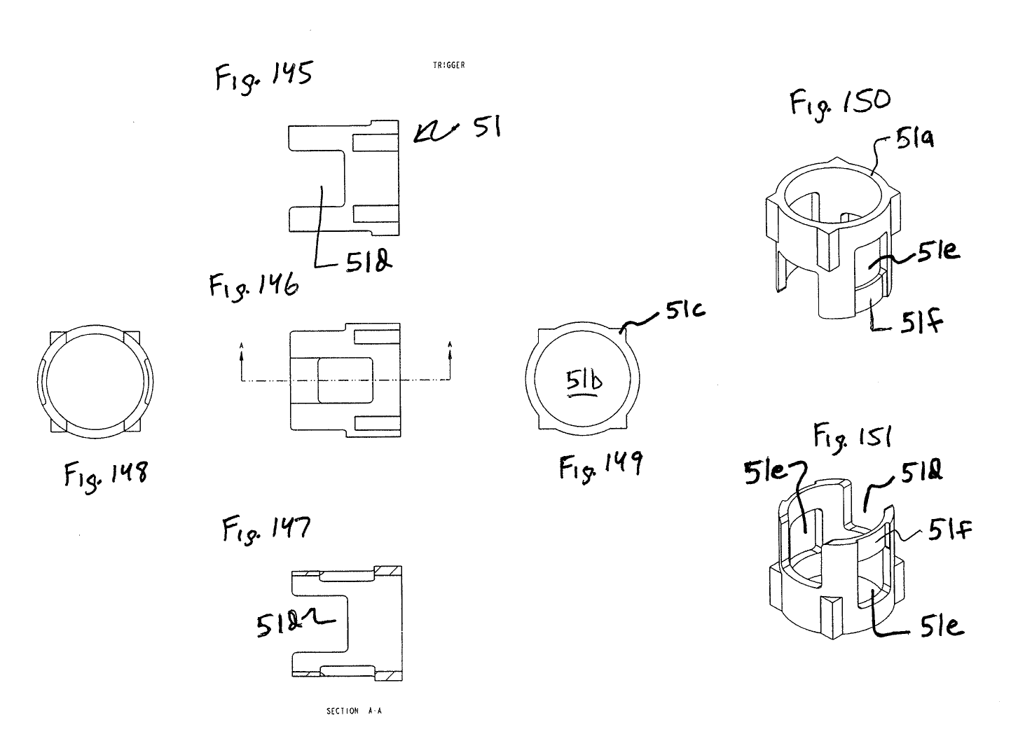

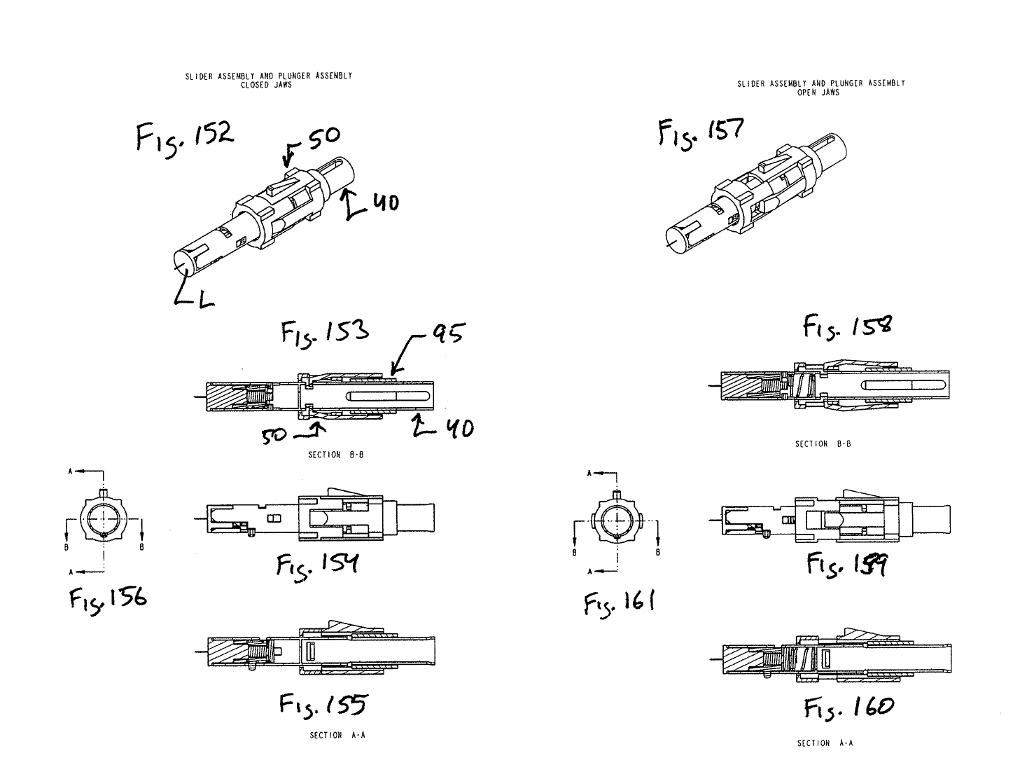

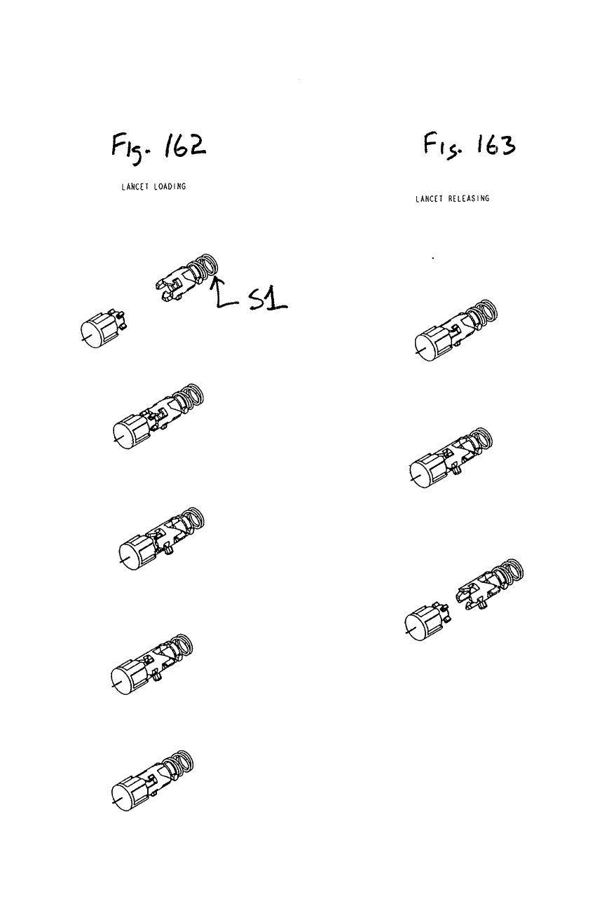

The proximal member 40 has a first end 41 and a second end 42 and includes two oppositely arranged recesses 40a which are sized and configured to receive therein the projections 52f of the member 52 (see FIGS. 126-161). A generally cylindrical internal space 40b is disposed within the member 40. In embodiments, the space 40b is sized and configured to receive therein a proximal portion 30d. A generally cylindrical outer surface 40c of the member 40 is sized and configured to slide within the assembly 50. This surface also receives thereon the sleeve 95 which abuts projections 40e. Once a proximal end 30d of the member 30 is inserted into a distal end of the member 40, two oppositely arranged deflectable projections 30e are sized and configured to slide within a respective slot of two oppositely arranged slots 40d of the member 40. The first end 41 is sized and configured to receive a lancet assembly L as will be discussed in detail herein. In this regards, an area of the first end 41 includes two oppositely arranged recesses 40f which are sized and configured to receive therein the projections 62a of the member 62 (see FIGS. 164 and 165) and serve to axially retain the member 62 within a proximal portion of the space 40b disposed between an internal wall 48g and the first end 41. The member 62 is inserted into this space after a spring S1 is first inserted so that the spring S1 becomes compressed between the member 62 and the wall 40g. Also arranged on the member 40 are four proximal slots 40h which are sized and configured to receive therein four projections L1 of the lancet L. One of these recesses 40h includes a releasable retaining recess 40i which is sized and configured to receive therein a rotatable and axially movable projection 63a of the member 63. Still further, the member 40 includes a lock recess 40j which is sixed and configured to receive therein a projecting portion of a lock member 81. When so engaged, the engagement between the lock 81 and the recess 40j prevents axial movement of the assembly 40 within the body 1.

With reference to FIGS. 118-122 and 162-165, the lancet assembly, in accordance with one non-limiting embodiment, utilizes main components L, 62, 63 and spring S1. A first component is a lancet L. This component L is a removable lancet having a needle N and that can be removed from the holder assembly or member 40 and can be replaced with a new one after use. A second component is a proximal member 63. This component is designed to remain within the assembly 40 and can rotate and move axially within the assembly 40. A third component is a distal member 62. This component is designed to remain within the assembly 40 and does not rotate, but can move slightly axially within the assembly 40. A spring S1 is configured to bias the member 62 toward the member 63 and functions to maintain the locking position of the lancet L, but also allows the member 63 to be rotated to the point where it can release from the assembly 40.

The lancet L has four projecting portions L1 which are sized and configured to be axially slide within the slots or recesses 40h of the member 40 (see FIGS. 92-97). Surfaces L2 are sized and configured to abut the fee ends of the member 40 when fully installed therein. A distal projecting portion L3 extends distally from a main body portion of the lancet L containing the projections L1. The distal portion L3 includes four projections L4 are sized and configured to engage in a releasable locking manner with four generally L-shaped locking recesses 63b of the member 63. As will be described in detail later on, the configuration of members 62, 63, L and S1 is such that a user cannot remove the lancet L from the end 41 of the assembly 40 until the member 63a is cause to rotate to the point that the locking recesses 63b release from the projections L4.

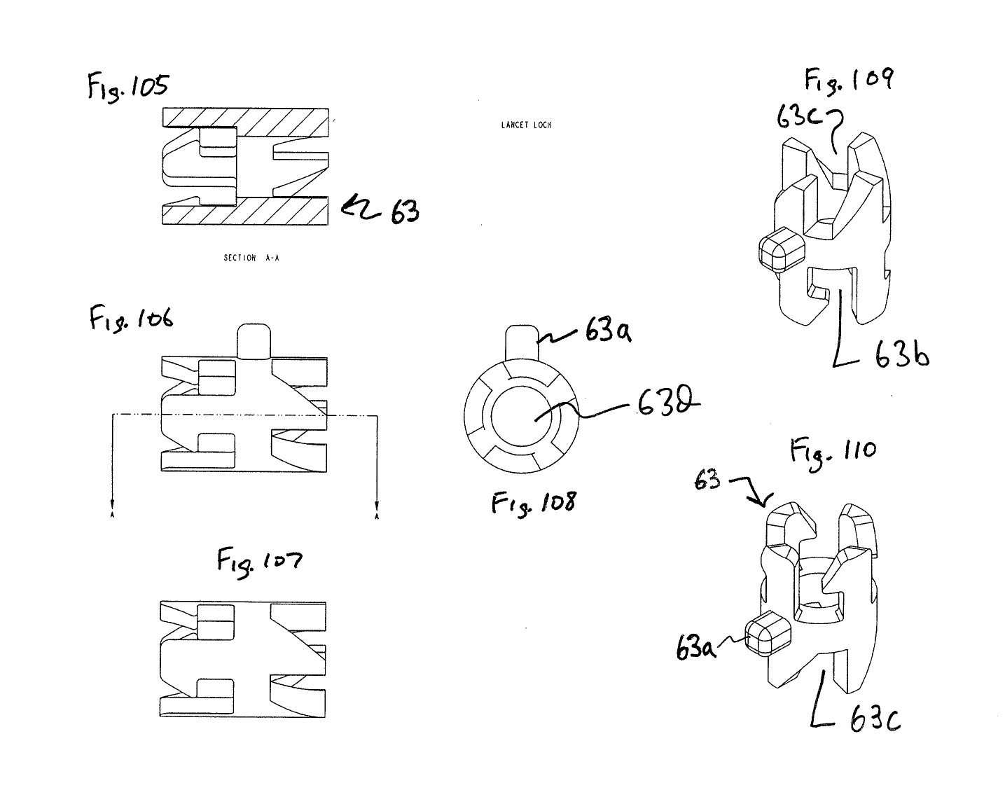

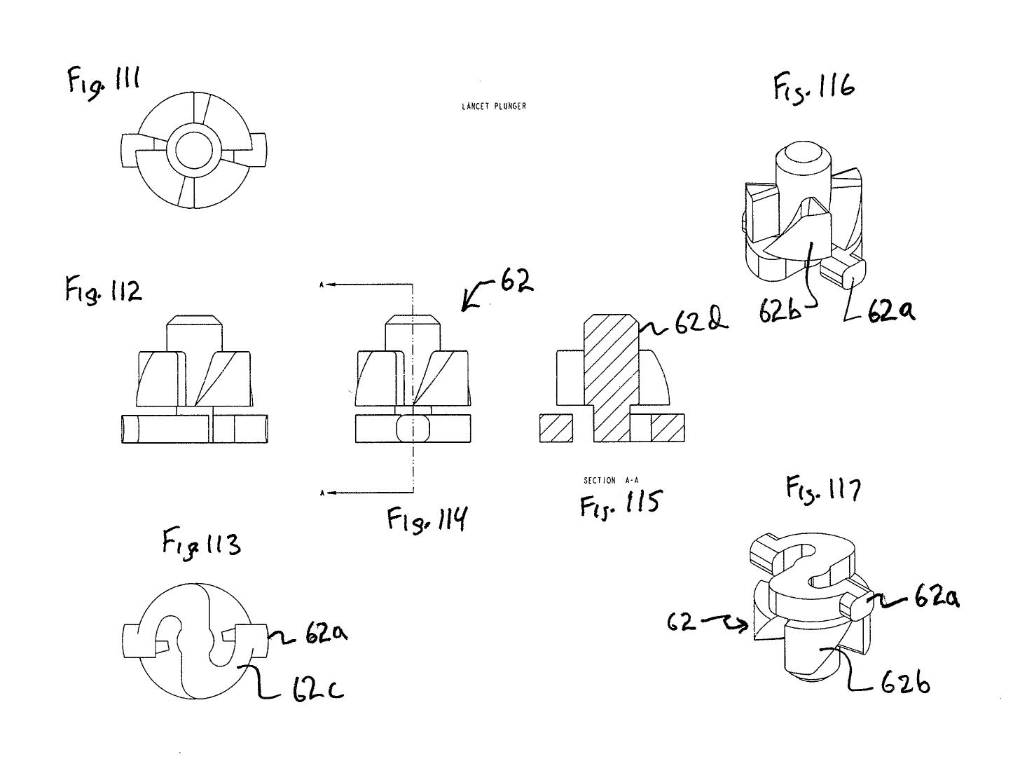

Referring to FIGS. 105-117, it can be seen that the member 63 has a main projection 63a that can be cause to rotate relative to the end 41 of the member 40 by the lancet release member 61. Four locking recesses 63b are sized and configured to receive therein the four projections L4 of the member L. Four tapered recesses 63c are sized and configured to rotatably and slidably axially engage with four comparably shaped projections 62b of the member 62. The surfaces of the recesses 63c and projections 62b are such that they under the expanding action or biasing force of the spring S1, the member 63 is maintained in the locking position. However, when the projection 63a is caused to rotate to a position allowing for release of the lancet L, the tapered surfaces of the recesses 63c slidably engage (via cam action) with the tapered surfaces of the projections 62b and cause compression of the spring S1 and movement of the projections 62a to a distal-most axial position of the slots 47f. The member 63 also includes a central recess 63b sized and configured to receive therein (to provide axial alignment) with a central projection 62d of the member 62. The projections 62a are arranged on two spiral shaped deflecting arms 62c which function as springs. Inward deflection of the arms 62c allows the member 62 to be inserted within the end 41. When properly inserted, the arms 62c deflect back to an original or relaxed position characterized by the projections 62a extending into the slots 40f.

With reference to FIGS. 126-161 it can be seen how the slider or movable sleeve assembly 50, in accordance with one non-limiting embodiment, utilizes main components 51 and 52. A first component is a front sleeve member 51 of the assembly 50. A second component is a rear or distal sleeve member 52 of the assembly 50.

The first component 51 is generally a sleeve shaped member having an annular front end 51a. A main through opening 51b extends from the end 51a and is sized and configured to receive therein the holding assembly 40 and allows it to move within it. Four corner projections 51c are arranged to linearly guide the member 51 in relation to the body 1. Each projection 51c has a surface which slidably engages with a surface in the body 1 so that the member 51 can slide axially back and forth within the body 1 without also rotating to any significant extent. Two oppositely arranged recesses 51d are arranged to linearly guide and receive two projections 52d of the member 52. Each projection 52d slidably engages with surfaces of the recess 51d in a manner which allows both members 51 and 52 to slide axially back and forth within the body 1 without also rotating to any significant extent relative to each other. Two oppositely arranged movement limiting recesses 51e are arranged to linearly guide and receive two arms 52e of the member 52. Each arm 52e has an inwardly projecting portion 52f which extends into one of the recesses 51e and slidably engages with surfaces of the recess 51e in a manner which allows both members 51 and 52 to slide axially back and forth (but to a limited extent) within the body 1 without also rotating to any significant extent relative to each other. When the lancet device LD is in the original position (see FIG. 7), the surface 51a contacts or abuts against surfaces 2k and 3i of the body 1.

The second component 52 is generally a sleeve shaped member having an annular rear end 52a. A main through opening 52b extends from the end 52a and is sized and configured to receive therein the holding member 40 and allows it to move within it. Four corner projections 52c are arranged to linearly guide the member 52 in relation to the body 1. Each projection 52c has a surface which slidably engages with a surface in the body 1 so that the member 52 can slide axially back and forth within the body 1 without also rotating to any significant extent. Two oppositely arranged projections 52d are linearly guided and are received within two recesses 51d of the member 51. Two projecting arms 52e slidably engage with surfaces of the recess 51e in a manner which allows both members 51 and 52 to slide axially back and forth within the body 1 without also rotating to any significant extent relative to each other. The oppositely arranged movement limiting recesses 51e are arranged to linearly guide and receive the two arms 52e of the member 52. Each arm 52e has an inwardly projecting portion 52f which extends into one of the recesses 51e and slidably engages with surfaces of the recess 51e in a manner which allows both members 51 and 52 to slide axially back and forth (but to a limited extent) within the body 1 without also rotating to any significant extent relative to each other. When the lancet device LD is in each of three main positions, the surface 52a contacts or abuts against a front end of the main spring S4. As will be described herein in detail, the act of placing the lancet device LD in the armed position, causes the member 52 to move axially away from the member 51. When this happens, the projecting portions 52f are cause to deflect outwardly and disengage from slots formed in the holder assembly 30/40. This outward deflection results from each arm 52e engaging with the surface 51f and, more specifically, when each bent portion 52g engages with a respective surface 51f.

When both members 51 and 52 are installed within the lancet device LD, the main spring S4 forces the member 52 towards member 51 so that the surface 51a contacts surface formed by surfaces 2k and 3i of the body 1. However, when a slide button 20 is moved distally to cause triggering of the lancet device LD (as shown in FIGS. 8 and 9), a projection 20c of the slide button 20 contacts a surface 52i of the projection 52h and causes the member 52 to move distally.

A lancet release system 60, in accordance with one non-limiting embodiment, is shown in FIGS. 164-172 includes a one-piece or integrally formed release button or member 61 which is biased toward an original position by a torsion spring S2. The button 61 includes a finger engaging surface 61a which is sized and configured to comfortably receive a thumb when the lancet device LD is properly grasped by a user. The button 61 has two oppositely arranged surfaces 61b which slidable engage with side surfaces of a groove formed in the body 1 (see FIGS. 5 and 6 as well as FIGS. 13 and 14). A connecting portion 61c extends into a circumferential slot 2e formed in the body 1 and connects an engaging portion 61d to the main portion of button 61. The engaging portion 61d is sized and configured to contact and cause rotational movement of the projection 63a and a corresponding rotation of member 63 during release and/or replacement of the lancet L. Another projecting portion 61e extends into another slot 2f of the body 1 and includes a biasing projection 61f. The projection 61f is sized and configured to extend into and engage with the bent portion S2b of the torsion spring S2. The other end S2c of the spring S2 is designed to contact a surface 2i/3j of the body 1. As a result, when the user rotates the button 61 to a lancet release position, the coils S2a of the spring S2 are cause to reduce in diameter slightly. When the button 61 is released, however, the spring S2 assumes an original more relaxed position and moves the button 61 back to an original position (see FIG. 6). The result is that the portion 61d no longer engages with the projection 63a thereby allowing the member 63 to rotate back to a locked position under the action of compression spring S1.

The basic operation of the lancet removal or replacement system will now be described with reference to FIGS. 105-122 and 163-172. Prior to use in forming a puncture and to the lancet device LD being made ready, and assuming that no lancet L is installed on the lancet device LD, the user can install a lancet L onto the front end of the member 40. To accomplish this, one typically first removes the front cap assembly 10. Doing so automatically results in the member 82 being caused to move forward under the action of the spring S3. This, in turn, causes the lock member 81 to pivot downward so that the end 81c enters into or engages with the slot 40j. This results in the holding member 40 being axially locked in place so that it cannot move axially backwards and forwards. The benefit of this is that the user can now install the lancet L without causing any axial movement of the holding member 40. Moreover, owing to the engagement between projections 30a and openings 2o/3o as well as that between the slots 40d and projections 30e, the holding member 40 is prevented from rotating relative to the body 1.

In one non-limiting manner, the user can rotate member 61 against the biasing force of torsion spring S2 to a release position that is between about 45 degrees to about 90 degrees from an original position. Next, the user grips a lancet L and slides it (distal end first) axially into the front end of the holding member 40. This is accomplished by aligning the projections L1 with the slots 40h. When the projections L4 reach an innermost point, the user can release the button 61 which will cause the member 63 to rotate to the point where the projections defined by recesses 63b to lockingly engage with the projections L4. Releasing the button 61 results in the force that was previously exerted on the projection 63a being reduced and allows the member to rotate to a locking position under the action of the spring S1 and member 62. When fully released, the slide 61 returns to an original position under the action of the spring S2 and the lancet L is locked to the member 40 so that it moves axially therewith and cannot be removed unless the lock system is undone.

Once a new lancet L is installed (see FIG. 162), the user can reinstall the cap assembly 10. When this happens, the member 82 is caused to move backwards and compresses the spring S3. This, in turn, causes the lock member 81 to pivot upward so that the end 81c exits out of or disengages from the slot 40j. This results in the holding member 40 being unlocked or freed so that it can now move axially backwards and forwards. Next, the user can, if desired, set a desired depth of penetration.

In order to install a new lancet L after use of the device LD, the user must first remove the old lancet L. This is accomplished by the user first removing the cap assembly 10 as discussed above. Next, the user can rotate member 61 against the biasing force of torsion spring S2 to a release position that is between about 45 degrees to about 90 degrees from an original position. This unlocks the old lancet L and allows it to be released or removed. At this point, the user grips the lancet L and slides it off the front end of the holding member 40 and preferably safely discards the same. The installation of a new lancet L can be accomplished in the manner noted above.

A lancet holding member lock system 70, in accordance with one non-limiting embodiment, is described in reference to FIGS. 186-201 and 203 and includes a one-piece or integrally formed pivoting lock member 81 which is biased toward a locking position by a compression spring S3. The member 81 includes a generally U-shaped mounting portion 81a, and a connecting portion 81b. An opening 81d allows the member 81 to be pivotally mounted to an axel portion 82a of ring-shaped member 82. The rod portion 81c is configured to engage with the slot 40j of the member 40. However, this locking engagement takes effect only when the front cover assembly 10 is removed from the lancet device LD. In this regard, the member 82 includes a body 82b having an inner annular space 82c which receives therein a portion of the spring S3. An opposite end of the spring S3 contacts a flange 2i/3k of the body 1. Two oppositely arranged projecting arms 82d protrude fowardly from the body 82b and have free ends 82e which can be contacted by annular surface 12j of member 12. When the front cap 10 is fully installed on the lancet device LD, the surface 12j contacts ends 82e and axially moves the member 82 distally. This action compresses the spring S3 and causes the member 81 to pivot out of engagement with the slot 40j of the holding assembly 40 thereby. In this unlocked state, the holding assembly 40 can be moved axially back and forth. However, when the cap assembly 10 is removed, the reverse happens and the lock member 81 is cause to pivot down and engage with the slot 40j of the assembly 40 and prevents axial movement thereof.

In accordance with one non-limiting embodiment of the invention there is provided a lancet device LD comprising a magnet member 90, a sleeve member 95 capable of being affected by magnetic forces emanating from the magnet member 90 and a movable lancet holder 40. At least one of the following is also utilized; a lancet releaser 61, 62 and/or 63 movable at least between an initial position and a position that allows a lancet L to be removed from the lancet holder 40 and a lancet holder lock 80 movable at least between a lock position that prevents axial movement of the lancet holder 40 along an axial position an unlock position that allows the lancet holder 40 to move along the axial position.

In embodiments, the lancet device further includes a slide button 20 movable at least between at least one of: an initial position to a cocking or arming position, a first position defined by a front portion of the slide button being generally adjacent a proximal end of the lancet device and a second position defined by a rear portion of the slide button being generally adjacent a distal end of the lancet device; an initial position to a triggering position; and an initial position to an arming and triggering position.

In embodiments, the lancet device further comprises an axially movable arming/triggering device 50 that, is movable at least between a first position and a second position.

In embodiments, the lancet holder lock 80 comprises at least one of: an axially movable member 82; a pivotally mounted projection 81 that engages with a recess or slot arranged on the lancet holder 40, at least one projection adapted to moved when a front cap 10 of the lancet device is installed; and a compression spring S3.

In accordance with one non-limiting embodiment of the invention there is provided a method of using the lancet device LD of any of the types described above, wherein the method comprises moving the sleeve member 95 from a first position surrounding the magnet member 90 to a second more distal position and after the lancet holder 40 moves to toward an extended position, moving the sleeve member 95 back to the first position surrounding the magnet member.

The device(s) described herein preferably utilizes one or more features disclosed in prior art documents expressly incorporated by reference herein. The documents expressly incorporated therein are hereby fully or entirely expressly incorporated by reference in the instant application. Furthermore, one or more of the various parts of the device can preferably be made as one-piece structures by e.g., injection molding, when doing so reduces costs of manufacture. One-piece elements can also be made as multiple piece elements. Non-limiting materials for most of the parts include synthetic resins such as those approved for lancet devices or other medical devices. Furthermore, the invention also contemplates that any or all disclosed features of one embodiment may be used on other disclosed embodiments, to the extent such modifications function for their intended purpose.

Materials that can by way of example be utilized include ABS (possibly PC/ABS) for the housing(s). One or more other components can be made of Delrin (Acetal). The magnet can be made of Neodymium, Grade 52. See http://www.kjmagnetics.com/proddetail.asp?prod=D46-N52. The springs can be spring steel. Finally, the metal sleeve can be made of zinc plated low carbon steel.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A lancet device comprising:
a magnet member;
a sleeve member capable of being affected by magnetic forces emanating from the magnet member;
a movable lancet holder; and
at least one of:
a lancet releaser movable at least between an initial position and a position that allows a lancet to be removed from the lancet holder; and
a lancet holder lock movable at least between a lock position that prevents axial movement of the lancet holder along an axial direction and an unlock position that allows the lancet holder to move along the axial direction.

2. The lancet device of claim 1, wherein the lancet holder is movable to a retracted position or a cocked position and an extended position or a piercing position.

3. The lancet device of claim 1, wherein the sleeve member is axially movable at least between:
an initial position and a retracted position; and
an extended position and the initial position.

4. The lancet device of claim 1, wherein the sleeve member is a generally cylindrical metal member.

5. The lancet device of claim 1, wherein the magnet member is a generally cylindrical permanent solid magnet member.

6. The lancet device of claim 1, further including: a slide button movable at least between at least one of:
an initial position to a cocking or arming position;
a first position defined by a front portion of the slide button being generally adjacent a proximal end of the lancet device and a second position defined by a rear portion of the slide button being generally adjacent a distal end of the lancet device;
an initial position to a triggering position; and
an initial position to an arming and triggering position.

7. The lancet device of claim 1, further comprising an axially movable arming/triggering device that is movable at least between a first position and a second position.

8. The lancet device of claim 7, wherein, in the first position, the arming/triggering device engages with a surface movable with the lancet holder, and, in the second position, disengages with the surface.

9. The lancet device of claim 7, wherein the arming/triggering device comprises a first member that is movable via a slide button and a second member with the first member and movable relative to the first member.

10. The lancet device of claim 1, further comprising an axially movable sleeve assembly that is movable at least between a first position and a second position.

11. The lancet device of claim 10, wherein, in the first position, at least one projection of the sleeve assembly engages with a surface movable with the lancet holder, and, in the second position, disengages with the surface.

12. The lancet device of claim 10, wherein the sleeve assembly comprises a first member that is movable via a slide button and a second member with the first member and movable relative to the first member.

13. The lancet device of claim 1, wherein the magnet member is at least one of:
axially retained;
fixed in place within the body;
has an axial length less than the axial length of the sleeve member; and
is coupled to the body via a member.

14. The lancet device of claim 1, wherein the lancet device comprises each of:
the lancet releaser movable at least between an initial position and a position that allows the lancet to be removed from the lancet holder; and
the lancet holder lock movable at least between a lock position that prevents axial movement of the lancet holder along an axial position an unlock position that allows the lancet holder to move along the axial position.

15. The lancet device of claim 1, wherein the lancet releaser comprises at least one of:
a slide member that can move at least partially circumferentially;
a projection that engages with a projection arranged on the lancet holder; and
a torsion spring.

16. The lancet device of claim 1, wherein the lancet holder lock comprises at least one of:
an axially movable member;
a pivotally mounted projection that engages with a recess or slot arranged on the lancet holder;
a projection adapted to move substantially perpendicular to an axis of the lancet holder that engages with a recess or slot arranged on the lancet holder;
at least one projection adapted to moved when a front cap of the lancet device is installed; and
a compression spring.

17. A method of using the lancet device of claim 1, the method comprising:
moving the sleeve member from a first position surrounding the magnet member to a second more distal position; and
after the lancet holder moves to toward an extended position, moving the sleeve member back to the first position surrounding the magnet member.

18. A method of using the lancet device of claim 1, the method comprising:
manually moving the sleeve member from a first position surrounding the magnet member to a second more distal position; and
after the lancet holder moves to toward an extended position, automatically moving the sleeve member back to the first position surrounding the magnet member.

19. A lancet device comprising:
an axially fixed magnet member;
a movable sleeve member capable of being affected by magnetic forces emanating from the magnet member;
a movable lancet holder;
a slide button movable between an initial position and a position causing both a cocking of the lancet device and a triggering of the lancet device; and
a lancet holder lock movable at least between a lock position that prevents axial movement of the lancet holder along an axial direction and an unlock position that allows the lancet holder to move along the axial direction.

20. A lancet device comprising:
an axially fixed magnet member;
a movable sleeve member capable of being affected by magnetic forces emanating from the magnet member;
a movable lancet holder;
a slide button movable between an initial position and a position causing both a cocking of the lancet device and a triggering of the lancet device,
a lancet releaser movable at least between an initial position and a position that allows a lancet to be removed from the lancet holder; and
a lancet holder lock movable at least between a lock position that prevents axial movement of the lancet holder along an axial direction and an unlock position that allows the lancet holder to move along the axial direction.
